(19) United States
(12) Patent Application Publication
(10) Pub. No.: US 2018/0064403 A1
(43) Pub. Date: Mar. 8, 2018

(54) MEDICAL IMAGE DIAGNOSTIC APPARATUS

(71) Applicant: Toshiba Medical Systems Corporation, Otawara-shi (JP)

(72) Inventors: Hidetaka KONTA, Utsunomiya (JP); Takeshi Obara, Otawara (JP); Masashi Ookawa, Nasushiobara (JP); Kiyomi Oshima, Nasushiobara (JP); Hideyuki Ooba, Nasushiobara (JP); Kentarou Furuhata, Otawara (JP); Yutaka Fukushiwa, Nasushiobara (JP)

(73) Assignee: Toshiba Medical Systems Corporation, Otawara-shi (JP)

(21) Appl. No.: 15/681,536

(22) Filed: Aug. 21, 2017

(30) Foreign Application Priority Data
Sep. 6, 2016 (JP) 2016-173635
Aug. 14, 2017 (JP) 2017-156375

(51) Int. Cl. A61B 5/00; A61B 5/055; A61B 6/03; A61B 6/00; A61B 8/00

(52) U.S. Cl. CPC A61B 5/743; A61B 5/055; G06T 11/206; A61B 6/463; A61B 8/463; A61B 6/032

(57) ABSTRACT

According to one embodiment, a medical image diagnostic apparatus includes a display and processing circuitry. The display displays a relationship diagram indicating a relationship between values which are respectively set for imaging parameters relating to an imaging condition of medical imaging. The processing circuitry updates the relationship diagram in accordance with a moving operation of a point corresponding to each of the values on the relationship diagram, or a change operation of the imaging condition. The processing circuitry changes the imaging condition before the moving operation or the change operation to an imaging condition which reflects the moving operation or the change operation.

### CROSS-REFERENCE TO RELATED APPLICATIONS

This application is based upon and claims the benefit of priority from the prior Japanese Patent Applications No. 2016-173635, filed Sep. 6, 2016, and No. 2017-156375, filed Aug. 14, 2017, the entire contents of both of which are incorporated herein by reference.

### FIELD

Embodiments described herein relate generally to a medical image diagnostic apparatus.

### BACKGROUND

Conventionally, for example, in the edit of an imaging condition in a magnetic resonance imaging apparatus, there is known a function of automatically calculating, at a time of changing an imaging parameter, the setting ranges of other related imaging parameters, and adjusting the other related imaging parameters such that these imaging parameters fall within the calculated setting ranges. The imaging condition is edited, as needed, for optimizing the imaging parameters.

The object is to intuitively present to an operator the influence which the edited imaging condition exerts on the imaging, at the time of editing the imaging condition.

### BRIEF DESCRIPTION OF THE DRAWINGS

FIG. 1 is a block diagram illustrating an example of the configuration of a magnetic resonance imaging apparatus according to a first embodiment.

FIG. 2 is a flowchart illustrating an example of a process procedure of an operation relating to the first embodiment.

FIG. 3 is a flowchart illustrating an example of the process procedure of the operation relating to the first embodiment.

FIG. 4 is a view illustrating an example of an edit screen and a relationship diagram which are displayed on a display in the first embodiment.

FIG. 5 is a view illustrating an example of a relationship diagram in which a fixing instruction is input with respect to an imaging parameter A in a modification of the first embodiment.

FIG. 6 is a view illustrating an example of imaging indices, desired edits of imaging indices, and imaging parameters relating to imaging indices in a second embodiment.

FIG. 7 is a flowchart illustrating an example of a process procedure of an operation relating to the second embodiment.

FIG. 8 is a flowchart illustrating an example of the process procedure of the operation relating to the second embodiment.

FIG. 9 is a view illustrating an example of an edit screen and a relationship diagram which are displayed on the display in the second embodiment.

FIG. 10 is a view illustrating an example of a relationship diagram in which a fixing instruction is input with respective to an imaging time which is an imaging index in a modification of the second embodiment.

FIG. 11 is a view illustrating an example of an edit screen and a relationship diagram, which are displayed on the display in a third embodiment.

FIG. 12 is a view illustrating an example in which adjustable ranges are superimposed on a radar chart, in a case of using the radar chart as a relationship diagram relating to a first applied example.

FIG. 13 is a view illustrating an example in which adjustable ranges are superimposed on a bar graph, in a case of using the bar graph as a relationship diagram relating to a first applied example.

FIG. 14 is a view illustrating an example of a case of using a tetrahedron as a relationship diagram relating to a third applied example.

FIG. 15 is a block diagram illustrating an example of the configuration of a medical image diagnostic apparatus according to a fourth applied example.

### DETAILED DESCRIPTION

In general, according to one embodiment, a medical image diagnostic apparatus includes a display and processing circuitry. The display is configured to display a relationship diagram indicating a relationship between a plurality of values which are respectively set for a plurality of imaging parameters relating to an imaging condition of medical imaging. The processing circuitry is configured to update the relationship diagram in accordance with a moving operation of a point corresponding to each of the values on the relationship diagram, or a change operation of the imaging condition, and configured to change the imaging condition before the moving operation or before the change operation to an imaging condition which reflects the moving operation or the change operation.

Embodiments will be described hereinafter with reference to the accompanying drawings. Incidentally, the structural elements having substantially the same structure are denoted by like reference numerals, and an overlapping description will be given only where necessary.

### First Embodiment

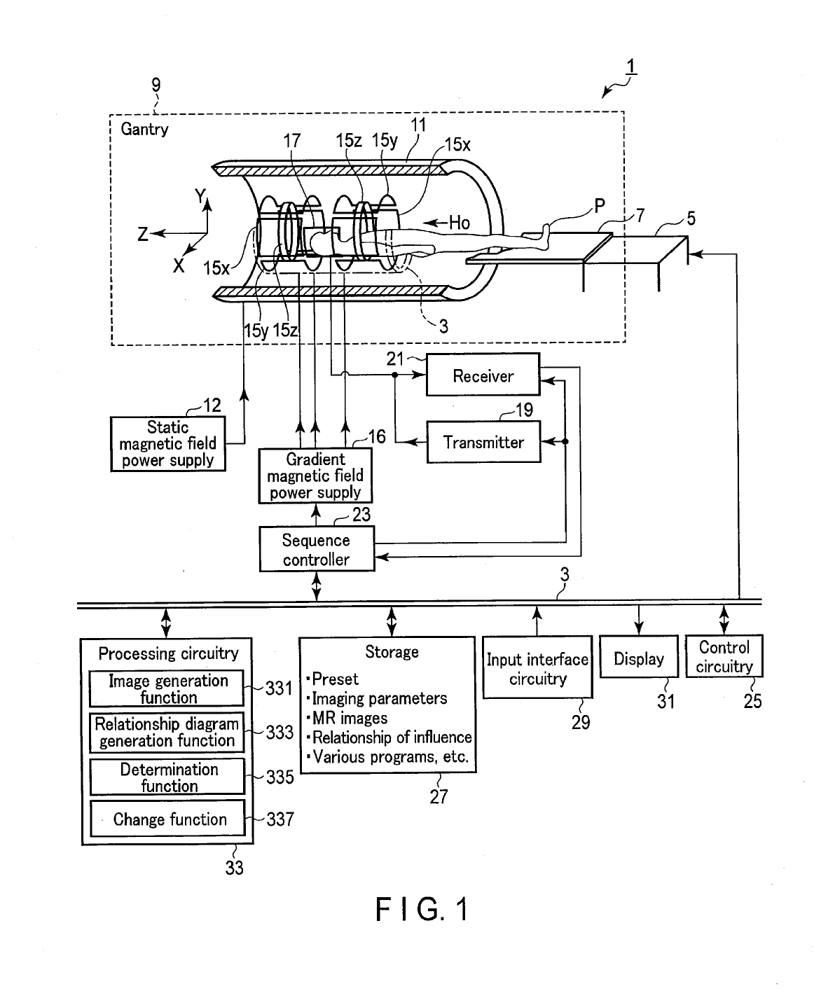

FIG. 1 is a block diagram illustrating the configuration of a magnetic resonance imaging (MRI) apparatus 1 according to the first embodiment. The MRI apparatus 1 includes a couch 5, a couch top 7, a gantry 9, a static magnetic field power supply 12, a gradient magnetic field power supply 16, a transmitter 19, a receiver 21 a sequence controller 23, a control circuitry 25, a storage 27, an input interface circuitry 29, a display 31, and a processing circuitry 33.

The couch 5 is installed such that the longitudinal axis of the couch 5 is parallel to the center axis of a static field magnet 11. The couch 5 drives the couch top 7 under the control by the control circuitry 25. The couch top 7 is moved in the longitudinal direction and in an up-and-down direction. In the state in which a subject P is placed on the couch top 7, the couch top 7 is inserted into a bore which is formed in the gantry 9.

The gantry 9 includes the static field magnet 11, a gradient coil 15, and an RF coil 17. The static field magnet 11 is a superconducting magnet by a superconducting coil, and is formed in a cylindrical shape. The static field magnet 11 forms a static magnetic field in an imaging space, by an electric current which is supplied from the static magnetic field power supply 12.

The gradient magnetic field power supply 16 supplies a pulse current, which relates to the generation of a gradient magnetic field, to the gradient coil 15. The gradient coil 15 is provided on the inside of the static field magnet 11 in the gantry 9. The gradient coil 15 includes three coils 15x, 15y and 15z which correspond to mutually orthogonal X, Y and Z axes. These three coils generate gradient magnetic fields by the supply of current from the gradient magnetic field power supply 16. A Z-axis direction is, for example, the longitudinal direction of the couch 5. In addition, a Y-axis direction is a vertical direction, and an X-axis direction is a direction perpendicular to the Z axis and Y axis.

The transmitter 19 supplies radio-frequency pulses, which correspond to the Larmor frequency for causing nuclear magnetic resonance, to the RF coil 17. The RF coil 17 receives the supply of radio-frequency pulses from the transmitter 19, and generates RF pulses. The RF pulses excite atomic nuclei in the subject P. The RF coil 17 receives an MR signal which is generated when the atomic nucleus in the subject P restores to the original state from the excited state. The receiver 21 applies various signal processes to the MR signal, and then generates echo data. The receiver 21 outputs the echo data to the sequence controller 23.

The sequence controller 23 includes a memory and a processor. The memory stores a preset which is read out from the storage 27. In the preset, a plurality of imaging protocols each having an imaging condition, which corresponds to a region of imaging and a purpose of imaging, are set in a desired order. The imaging condition is a combination of a plurality of imaging parameters. The imaging protocol is a unit of imaging, which is defined by setting imaging parameters. The sequence controller 23 controls the gradient magnetic field power supply 16, transmitter 19, etc. in accordance with the imaging protocol. The sequence controller 23 transfers the echo data to the processing circuitry 33. The control circuitry 25 controls the operation of the entirety of the MRI apparatus 1.

The storage 27 is composed of a hard disk drive, a solid-state drive, a magnetic disk, an optical disc, a semiconductor memory, etc. The storage 27 is a part of a storage unit. The storage 27 stores a pulse sequence, a preset, an imaging protocol, imaging parameters, etc. The storage 27 stores an operator's instruction via the input interface circuitry 29, MR images, various data received via a network, programs which are executed by various kinds of devices, and an edit screen of imaging parameters which are displayed on the display 31 at a time of editing the imaging condition. The edit screen is a screen for inputting values of imaging parameters.

The imaging parameters include TR (repetition time); the number of times of data acquisition (NAQ); the number of slices; a matrix size; aliasing prevention; a acceleration factor of parallel imaging by a multi-coil array; TI (inversion time); Prepulse (number, angle); the number of segments; the number of shots; content of prescan (RGN (reception gain), phase correction); an imaging parameter (asymmetric Fourier imaging (AFI) which utilizes complex conjugate symmetry; a repetition number (coverage number) of TR; a DWI axis number; Flip/Flop angle; TE (echo time); FOV (Field of View); Thickness (slice thickness); Band width (receiver coil); a region of imaging; time intervals (ETS (echo train spacing) of pulses) between echo trains in pulse sequences of EPI or FASE; B1 Shim Phase; a method (fine reconstruction: Fine Recon) for enhancing an apparent in-plane resolution by executing zero filling on MR data; couch position; DWI strength; kind of sequence (sequence type: RF Type, GR Type, etc.); PE (phase encode) direction; resolution; coverage division number; dB/dt; etc. Incidentally, the aliasing prevention may be read as, for example, an aliasing prevention coefficient. In the meantime, the imaging parameters are not limited to the above.

The input interface circuitry 29 inputs various instructions, commands, information, selection and settings from the operator to the MRI apparatus 1. The input interface circuitry 29 is realized by a mouse, a keyboard, a microphone, etc. The input interface circuitry 29 is an example of an input unit. The input interface circuitry 29 converts an input operation, which is received from the operator, to an electric signal, and outputs the converted electric signal to various devices. Incidentally, in the present specification, the input interface circuitry 29 is not limited to a device including physical operational components such as a mouse and a keyboard. For instance, examples of the input interface circuitry 29 include electric signal processing circuitry which receives an electric signal corresponding to an input operation from an external input device provided separately from the MRI apparatus 1, and outputs this received electric signal to the control circuitry 25 and processing circuitry 33.

The display 31 displays various images, an edit screen of values of imaging parameters, etc., under the control by the control circuitry 25. The display 31 is an example of a display unit.

The processing circuitry 33 includes a processor and a memory. The processing circuitry 33 executes various programs for executing respective processes in the MRI apparatus 1, thereby realizing functions corresponding to the programs. The processing circuitry 33 reads out a preset, which is selected by the operator, from the storage 27, and outputs the preset to the sequence controller 23, thereby executing data acquisition. The processing circuitry 33 executes, by an image generation function 331, two-dimensional or three-dimensional Fourier transform on the acquired echo data, etc., thereby generating an MR image. The processing circuitry 33 executes various image processes on the MR image. The image generation function 331 of the processing circuitry 33 is an example of an image generation unit.

The term “processor” used in the above description means, for example, a CPU (Central Processing Unit), a GPU (Graphics Processing Unit), or circuitry such as an ASIC (Application Specific Integrated Circuit), or a programmable logic device (e.g. SPLD (Simple Programmable Logic Device), CPLD (Complex Programmable Logic Device), FPGA (Field Programmable Gate Array)).

The processor realizes various functions by reading out and executing programs stored in the storage 27. In the meantime, instead of storing various programs in the storage 27, such a configuration may be adopted that the programs are directly incorporated in the circuitry in the processor in the control circuitry 25 or processing circuitry 33. In this case, the processor realizes the functions by reading out and executing the programs incorporated in the circuitry.

The configuration of the entirety of the MRI apparatus 1 according to the present embodiment has been described above. In this configuration, the MRI apparatus 1 according to the present embodiment includes the storage 27 functioning as the storage unit, a relationship diagram generation function 333 serving as a relationship diagram generation unit, and the display 31 functioning as the display unit. The storage 27 stores a plurality of imaging parameters in the imaging condition of magnetic resonance imaging, and data indicative of a relationship of influence between the imaging parameters. Based on a plurality of values corresponding to the imaging parameters and the relationship of influence, the relationship diagram generation function 333 generates a relationship diagram which visually indicates the relationship between the plural values. The display 31 displays the relationship diagram. Thus, according to the present embodiment, at a time of editing the imaging condition, it is possible to intuitively present to the operator the influence which the edited imaging condition exerts on imaging. Hereinafter, the MRI apparatus 1 according to the present embodiment will be described in detail.

The storage 27 according to this embodiment stores a plurality of parameters as an imaging condition of magnetic resonance imaging, and data indicative of the relationship of influence between the plural imaging parameters. The data indicative of the relationship of influence is, for example, a look-up table which shows the correspondency between a change amount of a certain imaging parameter and change amounts of the other imaging parameters. Incidentally, the storage 27 may store, instead of the look-up table, a calculation formula for calculating the change amounts of the other imaging parameters in relation to the change amount of the certain imaging parameter, as the data indicative of the relationship of influence. Besides, the data indicative of the relationship of influence may be information including a list showing restrictions (negative correlation, positive correlation, etc.) between a plurality of imaging parameters, and a weight indicating how a change amount of a certain imaging parameter influences the other imaging parameters.

Based on the plural values corresponding to the image parameters and the relationship of influence, the processing circuitry 33, which realizes the relationship diagram generation function 333, generates the relationship diagram which visually indicates the relationship between the plural values of imaging parameters. The processing circuitry 33 is an example of a relationship diagram generation unit. A graph, which is used as the relationship diagram, is, for example, a radar chart, a bar graph, etc. Incidentally, the kind and number of imaging parameters which are displayed on the relationship diagram, and the kind of the graph used as the relationship diagram, can be selected, as needed, by the operator.

The input interface circuitry 29 inputs an electric signal corresponding to a moving operation of a point indicative of the value of an imaging parameter on the relationship diagram (an input of movement of the point). The moving operation is an operation of moving the point in an increasing direction or a decreasing direction of the imaging parameter on the relationship diagram. The input interface circuitry 29 inputs a value of an imaging parameter on the edit screen. At this time, the input value of the imaging parameter is reflected on the relationship diagram by the processing circuitry 33. Specifically, the position of the point on the relationship diagram is moved in accordance with the input value of the imaging parameter.

Based on the change amount of the imaging parameter by the movement of the point, and based on the relationship of influence, the processing circuitry 33, which realizes a determination function 335, determines the values of the imaging parameters which co-vary with the movement of the point on the relationship diagram. In addition, based on the value of the imaging parameter, which is input on the edit screen, and based on the relationship of influence, the processing circuitry 33 determines the values of the imaging parameters which co-vary on the relationship diagram. The processing circuitry 33, which realizes the determination function 335, is an example of a determination unit.

The processing circuitry 33, which realizes a change function 337, changes, in accordance with a moving operation of the point indicative of the value of the imaging parameter, the imaging condition prior to the moving operation to an imaging condition which reflects the change amount of the imaging parameter by the movement of the point and the relationship of influence. At this time, the processing circuitry 33 updates the relationship diagram by changing the relationship between the values of the plural imaging parameters in the relationship diagram by using the determined values. In addition, the processing circuitry 33 changes, in accordance with an input operation of the value of the imaging parameter, the imaging condition prior to the input operation to an imaging condition which reflects the change amount of the imaging parameter after the input operation and the relationship of influence. In this manner, the input value of the imaging parameter is reflected on the relationship diagram. The processing circuitry 33, which realizes the change function 337, is an example of a change unit.

The display 31 displays the relationship diagram. Preferably, the display 31 displays the relationship diagram together with the edit screen of imaging parameters. Responding to the moving operation, the display 31 displays the updated relationship diagram. Hereinafter, in order to make the description more concrete, it is assumed that the relationship diagram is a radar chart. The number of axes of the radar chart corresponds to the total number of imaging parameters. Incidentally, the display 31 may display a plurality of radar charts in accordance with the number of imaging parameters.

(Operation)

The operation according to the present embodiment is an operation of updating and displaying the radar chart in response to the moving operation of the point indicative of the value of the imaging parameter on the radar chart, or in response to the input of the value of the imaging parameter on the edit screen. Hereinafter, the process procedure according to the present embodiment will be described.

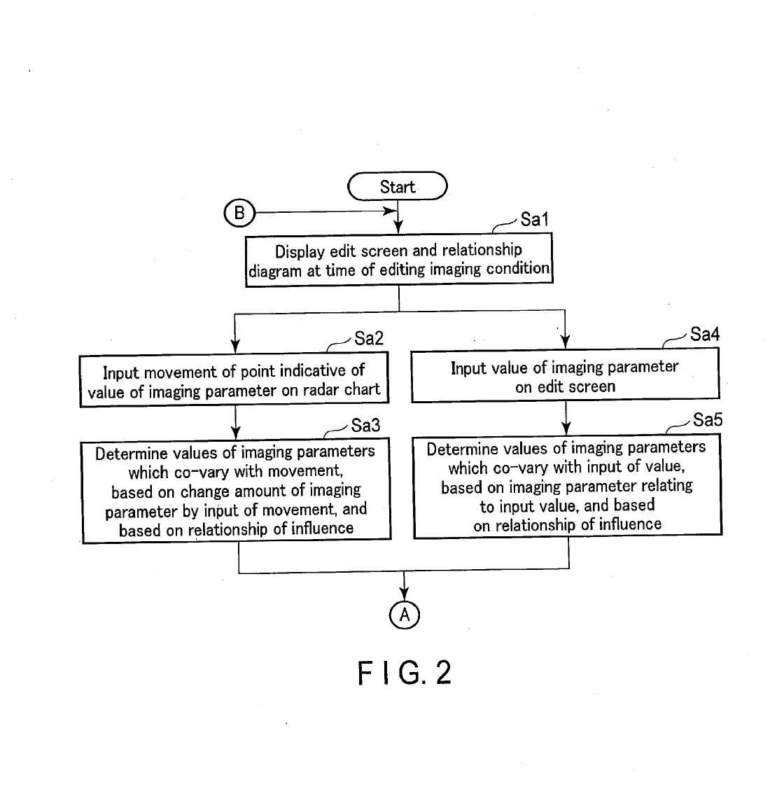

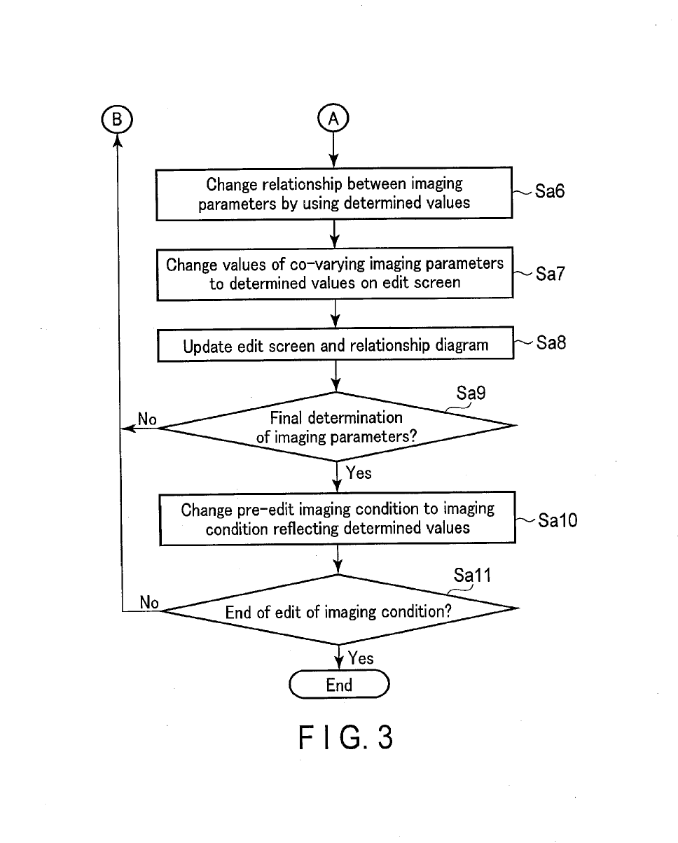

FIG. 2 and FIG. 3 are flowcharts illustrating the process procedure of the operation according to this embodiment.

At a time of editing the imaging condition, the edit screen and the radar chart are displayed (step Sa1). The radar chart displays, for example, imaging parameters included in a preset (or an imaging protocol) which is selected by the operator. The edit screen and radar chart may be switchably displayed as different screens.

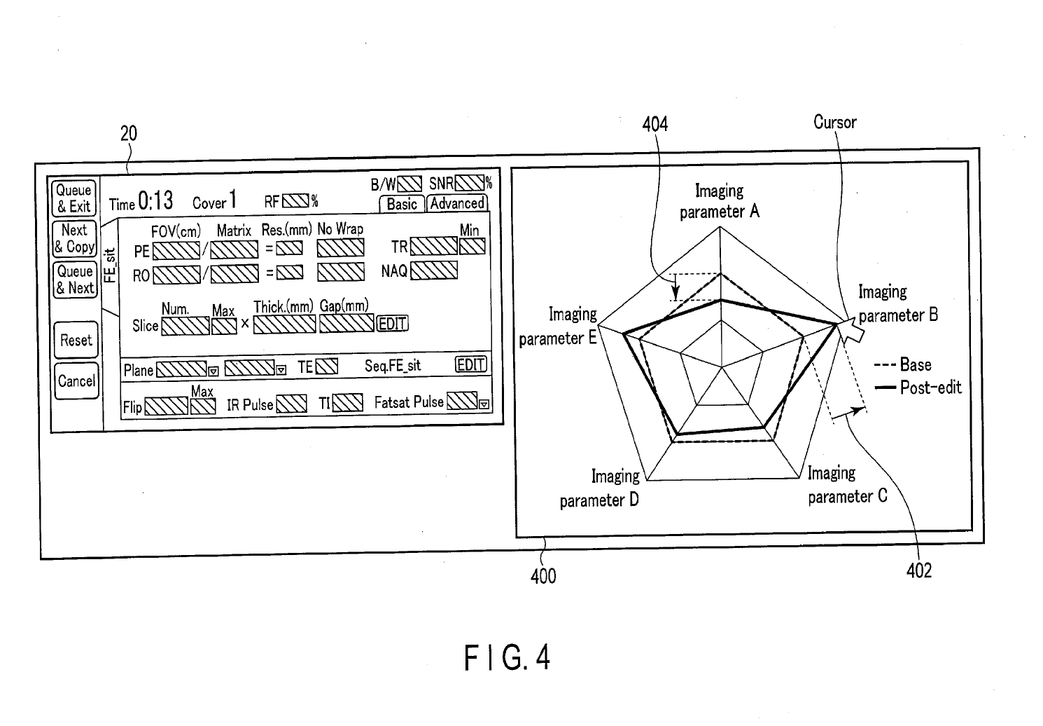

FIG. 4 is a view illustrating an edit screen 20 and a radar chart 400, which are displayed on the display 31. A plurality of locations of hatching on the edit screen of FIG. 4 are input areas of values of imaging parameters on the edit screen. As illustrated in FIG. 4, the edit screen 20 displays related information such as imaging contents and imaging protocol names. In the radar chart 400, the points indicative of the values of imaging parameters before edit, which correspond to imaging parameters A, B, C, D and E, are disposed equidistant from the center of the radar chart 400. The values of the imaging parameters before edit are, for example, values recommended by policies unique to a hospital in which the MRI apparatus 1 is installed, and recommended by the examination policy desired by the operator; values recommended by the supplier company of the MRI apparatus 1; or values of the result of the previous edit of the imaging condition. The values of the imaging parameter before edit correspond to reference values at the time of editing the imaging condition. Incidentally, the reference value may be a representative value (a mean value, a mode value, a median, etc.) of values of a plurality of identical imaging parameters which are commonly included in a plurality of imaging protocols, a plurality of presets, etc. which are selected by the operator. For example, a base of a dotted line shown in FIG. 4 is a polygonal line which connects points indicative of the reference values of the five imaging parameters in the radar chart 400, and corresponds to the relationship between the values of the five imaging parameters before edit.

In the radar chart 400, a movement of the point indicative of the value of the imaging parameter is input (step Sa2). In the meantime, when at least two of the plural imaging parameters, which are displayed on the radar chart 400, are set as targets of the moving operation, the points indicative of the values of the set imaging parameters are moved together by the moving operation. An arrow 402 shown in. FIG. 4 indicates a change amount of a point indicative of the value of the imaging parameter B (a change amount of the imaging parameter). The increase/decrease direction of the imaging parameter along the axis of the radar chart 400 can be set and changed, as needed, with respect to each of the imaging parameters. For example, when the imaging parameter is TR, it is preferable that the TR is as short as possible, from the standpoint of enhancement of a throughput. Thus, a direction away from the origin of the radar chart 400 is associated with the decreasing direction of TR.

Following the step Sa2, the values of the imaging parameters which co-vary with the movement of the point on the radar chart 400, are determined based on the change amount of the imaging parameter and the relationship of influence (step Sa3). Specifically, the values of the imaging parameters which co-vary with the movement of the point, are determined by using the change amount of the imaging parameter by the movement of the point and the look-up table. In the meantime, the values of the imaging parameters which co-vary with the movement of the point, may be determined by using the change amount of the imaging parameter and a calculation formula.

If the value of the imaging parameter is input on the edit screen (step Sa4), the values of the imaging parameters which co-vary with the input of the value, are determined on the radar chart 400, based on the imaging parameter relating to the input value and the relationship of influence (step Sa5). Specifically, the values of the imaging parameters which co-vary with the input, are determined by using the change amount of the imaging parameter corresponding to the input value, and the look-up table. In the meantime, the area, in which the value of the imaging parameter is input, is not limited to the edit screen, and may be, for example, an area (pull-down, dialog box, etc.) which is displayed when the cursor is moved to a vicinity of the imaging parameter on the radar chart.

In the radar chart 400, the positions of the points indicative of the values of the imaging parameters are changed by using the determined values, and the relationship between the values of the imaging parameters is also changed (step Sa6). In addition, in the edit screen, the values of the co-varying imaging parameters are changed to the determined values (step Sa7). In accordance with the change of the values of the imaging parameters, the edit screen and radar chart 400 are updated and displayed (step Sa8).

An arrow 404 in FIG. 4 indicates a movement of the point indicative of the value of the imaging parameter A, among the plural imaging parameters which co-vary with the movement of the point indicative of the value of the imaging parameter B. As illustrated in FIG. 4, the points indicative of the values of the imaging parameters A, C, D and E on the radar chart 400 are moved along the axes of the respective imaging parameters in the radar chart 400, in the co-variance with the movement of the point indicative of the value of the imaging parameter B. For example, a polygonal line that is a solid line in the radar chart 400 indicates the relationship between the values of the imaging parameters, which are changed in response to the movement of the point indicative of the value of the imaging parameter B. As illustrated in FIG. 4, the relationship between the values of the imaging parameters before and after the movement of the point, that is, the balance between the plural imaging parameters, is visualized by different display modes (e.g. pre-edit: dotted line, post-edit: solid line), and is displayed on the display 31. Although the relationship between the values of the imaging parameters before and after the movement of the point is discriminated by different kinds of lines, this relationship may be discriminated by different display modes, such as different hues, thicknesses, etc.

If the imaging parameters are not finally determined (No in step Sa9), the process of step Sa1 to Sa8 is repeated. If the imaging parameters are finally determined (Yes in step Sa9), the imaging condition before edit is changed to an imaging condition which reflects the values determined by the determination function 335 (step Sa10). Incidentally, the imaging condition, which is commonly included in a plurality of imaging protocol (imaging groups) that are associated by using the pulse sequence, purpose of imaging, region of imaging, etc., can be changed (batchwise conversion) to an imaging condition which reflects the change amount and the relationship of influence. In addition, a plurality of imaging groups may be prestored, and the batchwise conversion may be executed on an imaging group which is arbitrarily selected by the operator.

The imaging condition, which is changed by step Sa10, is stored in the storage 27. Until the edit of the imaging condition is finished, the process of step Sa1 to step Sa10 is repeated (No in step Sa11). When the imaging condition is re-edited (No in step Sa11), a normalization instruction, which makes uniform the positions of plural points in the radar chart 400, may be input. Specifically, in response to the input of the normalization instruction, the scale of each of the axes of the radar chart is changed. Thereby, the positions of plural points in the radar chart are uniformized and displayed. Incidentally, in the relationship diagram in FIG. 4, although the points indicative of the values of five imaging parameters are illustrated by way of example, the number of imaging parameters, which are displayed on the relationship diagram, is not limited to five. At this time, in the radar chart 400, the polygonal line that is the solid line, which indicates the relationship between the values of the imaging parameters, is displayed as a regular polygon by the normalization instruction.

According to the above-described configuration, the following advantageous effects can be obtained.

According to the MRI apparatus 1 relating to the present embodiment, a plurality of imaging parameters in an imaging condition of magnetic resonance imaging, and data indicative of a relationship of influence between the imaging parameters are stored. Based on a plurality of values corresponding to the imaging parameters and the relationship of influence, a relationship diagram, which visually indicates the relationship between the plural values, is generated and displayed. Accordingly, at the time of editing the imaging condition, it is possible to intuitively present to the operator the influence which the edited imaging condition exerts on the imaging.

For example, in accordance with the moving operation of the point indicative of the value of the imaging parameter on the relationship diagram, or in accordance with the input of the value of the imaging parameter on the edit screen, the positions of the points indicative of the values of the imaging parameters can be changed together with the relationship between the values of the imaging parameters, and can be displayed on the relationship diagram, and the values of the imaging parameters on the edit screen can be changed and displayed. In addition, according to the MRI apparatus 1, the polygonal line, which corresponds to the base before the moving operation of the point indicative of the value of the imaging parameter, can be displayed before and after the edit of the imaging parameter. Moreover, according to the MRI apparatus 1, in accordance with the normalization instruction, the positions of the plural points indicative of the values of the imaging parameters on the relationship diagram can be made uniform. Besides, according to the MRI apparatus 1, responding to the final determination of the values of the imaging parameters, the imaging condition, which is commonly included in the imaging groups, can be changed batchwise in accordance with the finally determined values of the imaging parameters.

From the above, according to the MRI apparatus 1 relating to the present embodiment, in the edit of the imaging condition, the changes of the values of the plural imaging parameters by the restrictions between the imaging parameters can be visually displayed on the relationship diagram, together with the relationship between the values of the imaging parameters and the base, and the change of the relationship between the values of the imaging parameters, relative to the base, can be intuitively presented to the operator. Specifically, according to the MRI apparatus 1, the operator is enabled to easily understand the changes of the imaging parameters, which are other than the edited imaging parameter and are changed by being influenced by the edited imaging parameter. From these matters, the re-setting of the imaging condition due to a change of the imaging parameter, which is not expected by the operator, can be reduced, and the edit efficiency of the imaging condition and the throughput of the MRI examination can be improved.

(Modification)

Compared to the first embodiment, the present modification is configured to restrict the movement of the point which co-vary in the radar chart. Accordingly, the input interface circuitry 29 inputs an instruction (restriction instruction) to restrict the influence between the imaging parameters due to the movement of the point, with respect to at least one of the plural imaging parameters displayed on the radar chart. The processing circuitry 33, which realizes the change function 337, changes the relationship of influence, in accordance with the restriction instruction to restrict the influence between the imaging parameters by the moving operation of the point. The processing circuitry 33, which realizes the determination function 335, determines the values of the co-varying imaging parameters, based on the changed relationship of influence and the change amount of the imaging parameter. The other configuration is the same as the configuration of the first embodiment.

The above-described restriction of movement is, for example, relaxation of movement and fixation. The relaxation of the influence between the imaging parameters due to the movement of the point will be first described, and then the fixation of the point on the radar chart will be described.

(Relaxation of the Influence Between Imaging Parameters)

The input interface circuitry 29 inputs, as a restriction instruction, a degree of priority which indicates the order of priority of relaxation of movement. As the degree of priority is lower, the influence between the imaging parameters becomes smaller. In the meantime, the input interface circuitry 29 may input a degree of priority with respect to the imaging parameter on the edit screen.

The processing circuitry 33, which realizes the change function 337, changes the relationship of influence in accordance with the degree of priority which is input as the restriction instruction. Concretely, the processing circuitry 33 decreases, in the look-up table, the change amount of the imaging parameter which is designated by the restriction instruction. Specifically, the processing circuitry 33 decreases, in the data indicative of the relationship of influence, the degree of influence on the imaging parameter designated by the restriction instruction, in accordance with the degree of priority. In addition, based on the degree of priority and the relationship of influence, the processing circuitry 33 increases the change amounts relating to the other imaging parameters for which the degree of priority is not designated, in order to harmonize the relationship of influence in accordance with the decrease of the change amount.

The processing circuitry 33, which realizes the determination function 335, determines the values of the imaging parameters which co-vary with the movement, based on the changed relationship of influence and the change amount of the imaging parameter by the movement of the point.

The display 31 displays, on the radar chart, a marker indicative of the degree of priority, at the point indicative of the value of the imaging parameter for which the degree of priority is designated. The marker indicative of the degree of priority is, for example, a predetermined graphic object with a numerical value indicative of the degree of priority.

The movement of the point relating to the imaging parameter, which is designated by the restriction instruction, becomes slow in the co-variance with the moving operation or the change operation. Specifically, the point relating to the imaging parameter, for which the restriction instruction is received, becomes less influenced by the moving operation or change operation, and thus becomes less easily movable on the radar chart. In addition, the moving operation for the imaging parameter, for which the restriction instruction is received, is suppressed, and the point relating to the imaging parameter, for which the restriction instruction is received, becomes difficult to move on the radar chart. The restriction instruction is useful when restrictions are to be imposed on the co-variance characteristic of the imaging parameter by the moving operation or change operation.

(Fixation of Imaging Parameters)

The input interface circuitry 29 inputs, as the restriction instruction, a fixing instruction to fix the point indicative of the value of the imaging parameter on the radar chart, with respect to at least one of the plural imaging parameters displayed on the radar chart. In the meantime, the input interface circuitry 29 may input the fixing instruction with respect to the imaging parameter on the edit screen.

The processing circuitry 33, which realizes the change function 337, changes the relationship of influence in accordance with the fixing instruction. Concretely, the processing circuitry 33 sets, in the look-up table, the change amount of the imaging parameter, which is designated by the restriction instruction, to zero. Specifically, the processing circuitry 33 sets to zero the degree of influence on the imaging parameter designated by the fixing instruction, in the data indicative of the relationship of influence. In addition, based on the imaging parameter designated by the fixing instruction and the relationship of influence, the processing circuitry 33 increases the change amounts relating to the other imaging parameters which are not designated by the fixing instruction, in order to harmonize the relationship of influence.

The processing circuitry 33, which realizes the determination function 335, determines the values of the other imaging parameters than the imaging parameter designated by the fixing instruction, based on the changed relationship of influence and the change amount of the imaging parameter by the movement of the point.

The display 31 displays, on the radar chart, a marker indicative of fixation, at the point indicative of the value of the imaging parameter designated by the fixing instruction. The marker indicative of fixation is, for example, a graphic object of a pin shape.

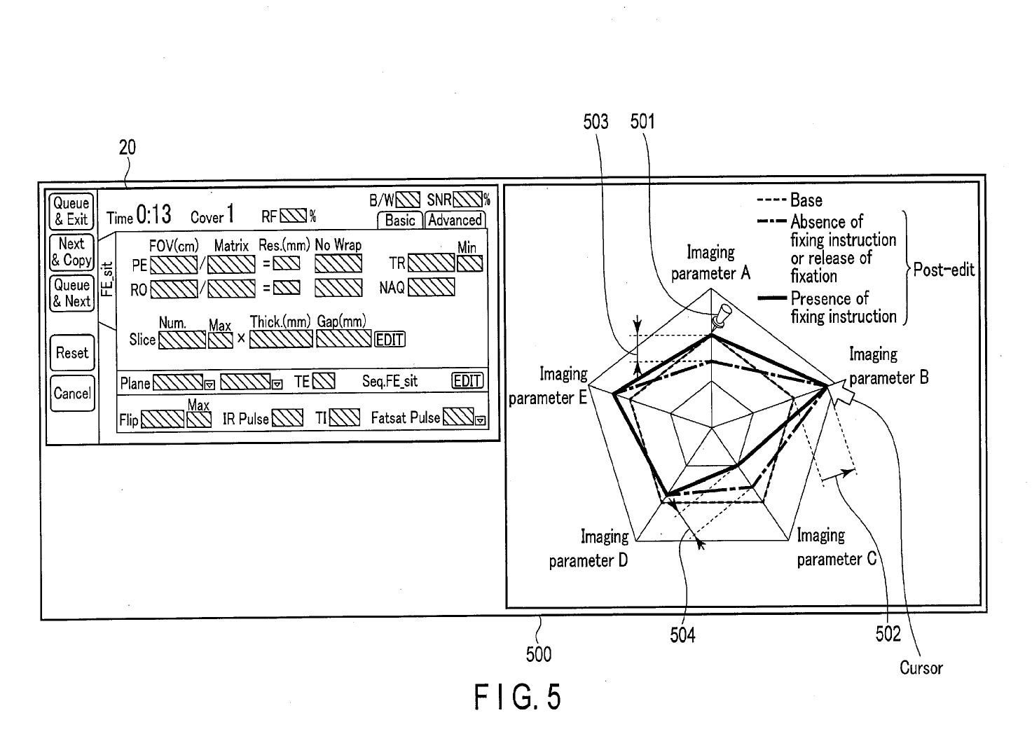

FIG. 5 is a view illustrating an example of a radar chart 500 in which the fixing instruction is input with respect to the imaging parameter A. A marker 501 indicative of fixation is displayed as a pin-shaped image at the point indicative of the value of the imaging parameter A in FIG. 5. As illustrated in FIG. 5, in a movement 502 of the point indicative of the value of the imaging parameter B, the point indicative of the value of the imaging parameter A remains fixed. Reference numeral 503 in the radar chart 500 indicates a difference between the position of the point indicative of the value of the imaging parameter A in the case of the absence of the fixing instruction, and the position of the point indicative of the value of the imaging parameter A in the case of the presence of the fixing instruction, in the movement 502 of the point indicative of the value of the imaging parameter B. Reference numeral 504 in the radar chart 500 indicates a movement amount of the point indicative of the value of the imaging parameter C in the case in which the imaging parameter A is fixed.

In the radar chart 500 in FIG. 5, when the point indicative of the value of the imaging parameter B is moved beyond a position indicative of a limit value of the imaging parameter (hereinafter referred to as “parameter limit position”), this point is moved back to the parameter limit position. Specifically, the movement of the point indicative of the value of the non-fixed imaging parameter is restricted by the parameter limit position. In the meantime, when the point indicative of the value of the imaging parameter B is moved beyond the parameter limit position, the marker 501 may be displayed in a different mode, for example, in a fallen pin shape, at a position apart from the position designated by the fixing instruction. At this time, the fixation of the point indicative of the value of the imaging parameter A is released, and character information notifying the release of the fixation of the point, for example, a character string, “Do you agree with the release of fixation?”, may be displayed.

According to the above-described configuration, the following advantageous effects can be obtained.

According to the MRI apparatus 1 relating to the present modification, the restriction instruction to restrict the influence between the imaging parameters due to the movement of the point is input with respect to the imaging parameter displayed on the relationship diagram. The relationship of influence is changed in accordance with the restriction instruction, and the values of the imaging parameters which co-vary with the movement, are determined based on the changed relationship of influence and the change amount of the imaging parameter. Accordingly, in addition to the advantageous effects of the first embodiment, at the time of editing the imaging condition in the case in which the influence between the imaging parameters is restricted, it is possible to intuitively present to the operator the influence which the edited imaging condition exerts on the imaging.

For example, in the relationship diagram, the movement of the point indicative of the value of the imaging parameter can be restricted. In addition, according to the present MRI apparatus 1, the marker can be displayed on the relationship diagram with respect to the imaging parameter for which the restriction of movement is designated. Thus, the operator can intuitively recognize, together with the relationship between the values of the imaging parameters, the state of change of the points indicative of the values of the other imaging parameters to which no priority is given.

In addition, according to the MRI apparatus 1, in the relationship diagram, the position of the point indicative of the value of the imaging parameter can be fixed such that this point does not move. Thereby, the operator can intuitively visually recognize, together with the relationship between the values of the imaging parameters, the state of change of the other imaging parameters due to the fixation of the imaging parameter. From these matters, according to the present MRI apparatus 1, a change of the imaging parameter, which the operator does not expect, can be prevented, re-setting of the imaging condition can be made needless, and the throughput of the edit of the imaging condition can be improved.

### Second Embodiment

Compared to the first embodiment, the present second embodiment is configured to move, on a relationship diagram, points indicative of values of a plurality of imaging indices which relate to a plurality of imaging parameters and which influence magnetic resonance imaging, and configured to display these points together with a relationship between the values of the imaging indices. Accordingly, based on the plural values corresponding to the plural imaging indices which relate to the imaging parameters and which influence magnetic resonance imaging, and based on the relationship of influence, the processing circuitry 33, which realizes the relationship diagram generation function 333, generates a relationship diagram which visually indicates the relationship between the plural values. The display 31 displays the relationship diagram. The other configuration of the second embodiment is the same as the configuration of the first embodiment.

The storage 27 according to the present embodiment stores a plurality of imaging indices. Each of the imaging indices is an index which indicates a degree of influence which the imaging condition exerts on imaging. Incidentally, the imaging index may be an index relating to an artifact. At this time, the value of the imaging index is a value indicative of the degree of the artifact. The storage 27 stores a conversion table which indicates a conversion relationship between the value of the imaging parameter and the value of the imaging index. Incidentally, the storage 27 may store, instead of the conversion table, a conversion formula which indicates the conversion relationship between the value of the imaging parameter and the value of the imaging index.

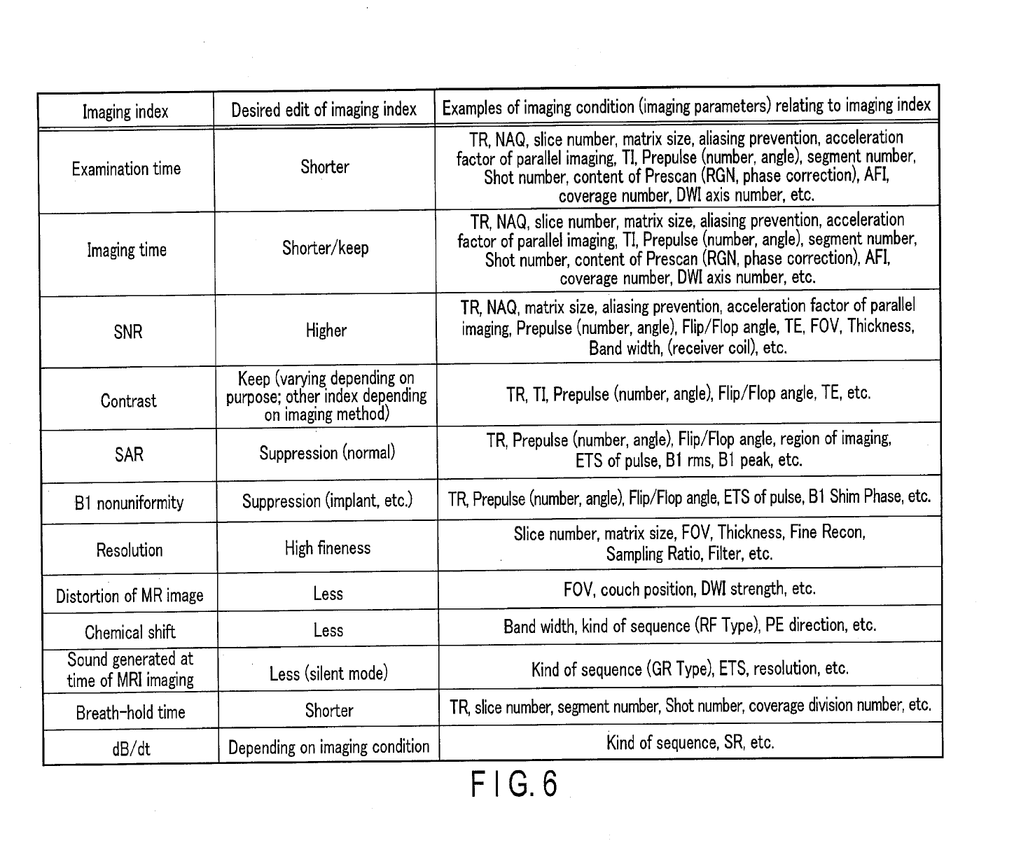

FIG. 6 is a view illustrating an example of the imaging indices, desired edits of the imaging indices, and the imaging parameters relating to the imaging indices. As illustrated in FIG. 6, each of the plural imaging indices is associated with the other imaging indices via imaging parameters. The values of the imaging indices are mutually associated via the relationship of influence between the imaging parameters and the conversion table.

Based on the plural values corresponding to the plural imaging indices which relate to the imaging parameters and which influence magnetic resonance imaging, and based on the relationship of influence, the processing circuitry 33, which realizes the relationship diagram generation function 333, generates the relationship diagram which visually indicates the relationship between the plural values. Incidentally, the kind and number of imaging indices, which are displayed on the relationship diagram, can be selected, as needed, by the operator.

The input interface circuitry 29 inputs an electric signal corresponding to a moving operation of a point indicative of the value of the imaging index on the relationship diagram (an input of movement of the point). The moving operation is an operation of moving the point in an increasing direction or a decreasing direction of the imaging index on the relationship diagram. The difference between the first embodiment and the second embodiment is that the objects indicated by the points in the relationship diagram are different.

Based on the change amount of the imaging parameter due to the change of the value of the imaging index by the movement of the point, and based on the relationship of influence, the processing circuitry 33, which realizes the determination function 335, determines the values of the imaging indices which co-vary with the movement of the point on the relationship diagram.

The processing circuitry 33, which realizes the change function 337, updates, in accordance with the moving operation of the point indicative of the value of the imaging index, the relationship diagram by changing the relationship between the values of the imaging indices on the relationship diagram by using the determined values of the imaging indices.

The display 31 displays the relationship diagram which visually indicates the relationship between the plural values corresponding to the plural imaging indices that relate to the imaging parameters and that indicate the influence on the magnetic resonance imaging. Hereinafter, in order to make the description more concrete, it is assumed that the relationship diagram is a radar chart. The number of axes of the radar chart corresponds to the total number of imaging indices. Incidentally, the display 31 may display a plurality of radar charts in accordance with the number of imaging indices.

(Operation)

The operation according to the present embodiment is an operation of updating and displaying the radar chart in response to the moving operation of the point indicative of the value of the imaging index on the radar chart, or in response to the input of the value of the imaging parameter on the edit screen. Hereinafter, the process procedure relating to the operation of the present embodiment will be described.

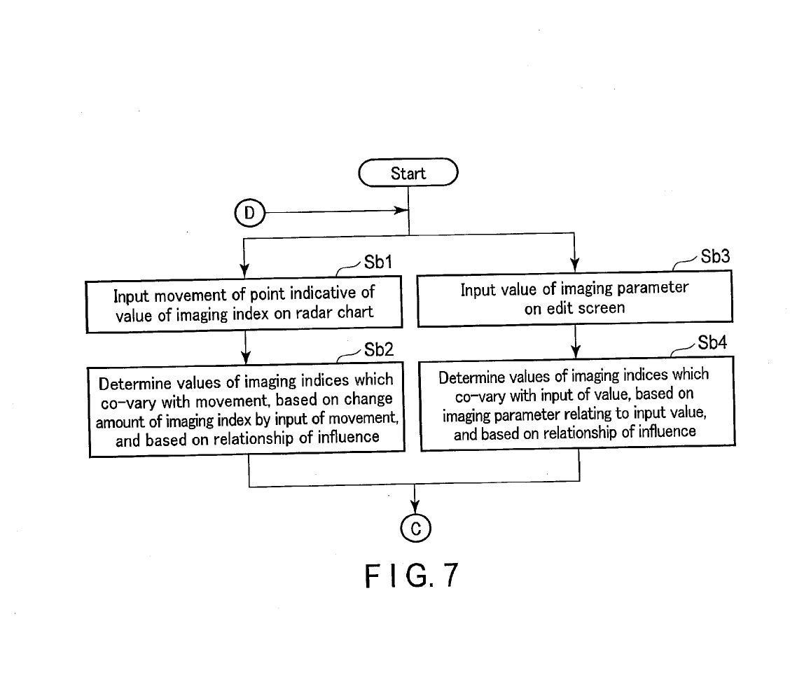

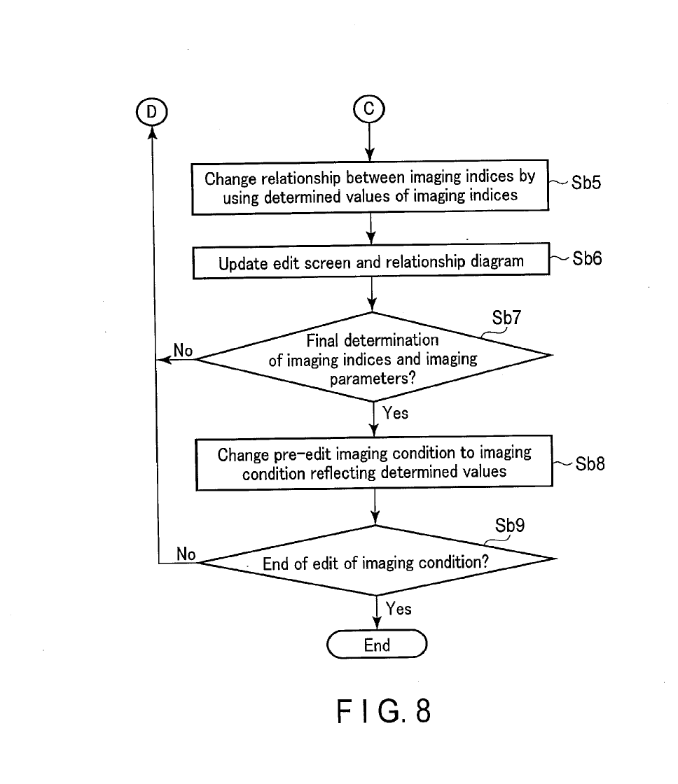

FIG. 7 and FIG. 8 are flowcharts illustrating the process procedure of the operation according to this embodiment.

The radar chart, which is displayed at the time of editing the imaging condition, displays, for example, imaging indices which are selected by the operator. Incidentally, the imaging indices may be newly created in accordance with plural imaging parameters designated by the operator. At this time, the values of the new imaging indices are determined by using the values of the plural imaging parameters designated by the operator, and the relationship of influence. In addition, a conversion relationship between the values of the new imaging indices and the values of the imaging parameters is newly created, for example, by the operator's instruction or by a predetermined learning function, and the created conversion relationship is added to the conversion table. In the meantime, instead of the conversion table, a conversion relationship between the values of the imaging parameters and the values of the imaging indices may be newly created by the operator's instruction or a predetermined learning function, and this conversion relationship may be newly stored in the storage 27 as a conversion formula.

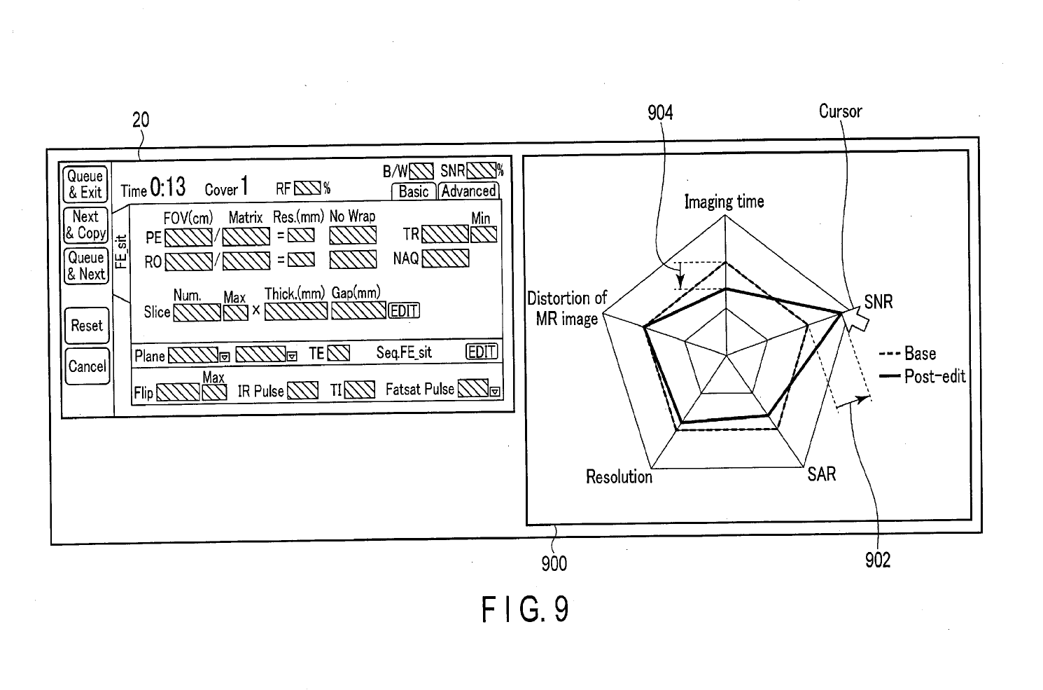

FIG. 9 is a view illustrating an edit screen 20 and a radar chart 900, which are displayed on the display 31. In the radar chart 900, the points indicative of the values of imaging indices before edit, which correspond to imaging indices (SNR, SAR, resolution, MR image distortion, and imaging time), are disposed equidistant from the center of the radar chart 900. The values of imaging indices before edit are, for example, values recommended by policies unique to the hospital in which the MRI apparatus 1 is installed, and recommended by the examination policy desired by the operator, values recommended by the supplier company of the MRI apparatus 1, etc. The values of the imaging indices before edit correspond to reference values at the time of editing the imaging condition. For example, a base of a dotted line shown in FIG. 9 is a polygonal line which connects points indicative of the reference values of the five imaging indices on the radar chart 900, and corresponds to the relationship between the values of the five imaging indices before edit. In the meantime, a reference image relating to the reference values of the imaging indices may be displayed on the display 31, together with the edit screen 20 and radar chart 900. The reference image corresponding to the reference values is stored in the storage 27.

In the radar chart 900, a movement of the point indicative of the value of the imaging index is input (step Sb1). In the meantime, when at least two of the plural imaging indices, which are displayed on the radar chart 900, are set as targets of the moving operation, the points indicative of the values of the set imaging indices are moved together by the moving operation. An arrow 902 shown in FIG. 9 indicates, in the radar chart 900, a change amount of a point indicative of the value of the imaging index: SNR (a change amount of the imaging index). The increase/decrease direction of the imaging index along the axis of the radar chart 900 can be set and changed, as needed, with respect to each of the imaging indices. For example, as illustrated in FIG. 6, when the imaging index is SNR, it is preferable that the SNR is as high as possible, from the standpoint of enhancement of image quality. Thus, a direction away from the origin of the radar chart 900 is associated with the increasing direction of SNR.

Following the step Sb1, the values of the imaging indices which co-vary with the movement of the point on the radar chart 900, are determined based on the change amount of the imaging index and the relationship of influence (step Sb2). Specifically, the change amounts of the imaging parameters which co-vary with the movement of the point, are determined by using the change amount of the value of the imaging index corresponding to the moved point, and the conversion table. Next, like the description in the first embodiment, the values of the imaging parameters, which are influenced, are determined based on the change amount of the imaging parameter and the relationship of influence. Subsequently, in the radar chart 900, the values of the imaging indices which co-vary with the movement of the point, are determined by using the determined values of the imaging parameters and the conversion table. Incidentally, the values of the co-varying imaging indices may be calculated by using the change amount of the imaging index corresponding to the moved point, a conversion formula, and a calculation formula. In the radar chart 900 in FIG. 9, if FIG. 6 is referred to, the imaging indices which co-vary with the movement of the point indicative of the value of the SNR, are the SAR, resolution, and imaging time.

As an example of the above-described determination of the values of the imaging indices, a case in which the value of SNR is increased by 5 (+5) will be described. In order to make the description simpler, it is assumed that the imaging parameters relating to the SNR are the TR and flip angle. At this time, the change amount of the TR and the change amount of the flip angle are determined by using the change amount (+5) of the SNR and the conversion table. Next, the values of the imaging parameters, which are influenced by the change of the TR, are determined based on the change amount of the TR and the relationship of influence. In addition, the values of the imaging parameters, which are influenced by the change of the flip angle, are determined based on the change amount of the flip angle and the relationship of influence. Next, the values of the imaging indices which co-vary with the movement of the point indicative of the value of the SNR, are determined by using the determined values of the imaging parameters and the conversion table. Incidentally, the values of the imaging indices which co-vary with the movement of the point indicative of the value of the SNR, may be determined by using the determined values of the imaging parameters and the conversion formula.

If the value of the imaging parameter is input on the edit screen (step Sb3), the values of the imaging indices which co-vary with the input of the value, are determined on the radar chart 900, based on the imaging parameter relating to the input value, and the relationship of influence (step Sb4). Specifically, the values of the imaging parameters, which are influenced by the input of the value of the imaging parameter, are first determined by using the change amount of the imaging parameter corresponding to the input value, and the look-up table. Next, the values of the imaging indices which co-vary with the input of the value of the imaging parameter, are determined on the radar chart 900 by using the determined values of the imaging parameters, and the conversion table.

In the radar chart 900, the positions of the points indicative of the values of the imaging indices are changed by using the determined values of the imaging indices, and the relationship between the values of the imaging indices is also changed (step Sb5). In addition, the values of the imaging parameters on the edit screen are changed based on the determined values of the imaging indices and the conversion table. In accordance with the change of the values of the imaging indices and the values of the imaging parameters, the edit screen 20 and radar chart 900 are updated and displayed (step Sb6). In FIG. 9, the imaging index “MR image distortion” has no relation to the movement of the point indicative of the value of the imaging index “SNR”. Thus, in the update of the radar chart 900, the point indicative of the value of the MR image distortion is unchanged.

An arrow 904 in FIG. 9 indicates a movement of the point indicative of the value of the imaging time, among the plural imaging indices which co-vary with the movement of the point indicative of the value of the SNR. As illustrated in FIG. 9, the points indicative of the values of the SAR, resolution and imaging time on the radar chart 900 are moved along the axes of the radar chart 900, in the co-variance with the movement of the point indicative of the value of the SNR. For example, a polygonal line that is a solid line in the radar chart 900 indicates the relationship between the values of the imaging indices, which are changed in response to the movement of the point indicative of the value of the SNR. As illustrated in FIG. 9, the relationship between the values of the imaging indices before and after the movement of the point, that is, the balance between the plural imaging indices, is visualized by different display modes (e.g. pre-edit: dotted line, post-edit: solid line), and is displayed on the display 31.

In the meantime, at the time of updating the edit screen 20 and radar chart 900, a tentative image may be displayed together with the updated edit screen 20 and radar chart 900. The tentative image is generated in the process of step Sb6, based on the imaging condition relating to the changed imaging indices, and the reference image. The tentative image is an image which tentatively reflects an imaging result relating to the imaging indices on the updated radar chart 900. For example, when the value of the SNR is larger than the reference value, the tentative image becomes an image with lower noise than the reference image. In addition, when the value of the resolution is larger than the reference value, the tentative image becomes an image with higher fineness than the reference image.

If the imaging indices and imaging parameters are not finally determined (No in step Sb7), the process of step Sbl to Sb6 is repeated. If the imaging indices and imaging parameters are finally determined (Yes in step Sb7), the imaging condition before edit is changed to an imaging condition which reflects the values determined by the determination function 335 (step Sb8). Until the edit of the imaging condition is finished, the process of step Sb1 to step Sb7 is repeated (No in step Sb9).

When the imaging condition is re-edited (No in step Sb9), a normalization instruction, which makes uniform the positions of plural points on the radar chart 900, may be input. Specifically, in response to the input of the normalization instruction, the scale of each of the axes of the radar chart is changed. Thereby, the plural points in the radar chart are made uniform and displayed. For example, in the radar chart 900, the polygonal line that is the solid line, which indicates the relationship between the values of the imaging indices, is displayed as a regular polygon by the normalization instruction.

In the meantime, when the imaging condition is re-edited (No in step Sb9), the tentative image, which is generated in accordance with the moving operation, may be displayed as a reference image, together with the updated edit screen 20 and radar chart 900. At this time, the generated tentative image is updated as a new reference image, and is stored.

According to the above-described configuration, the following advantageous effects can be obtained.

According to the MRI apparatus 1 relating to the present embodiment, a plurality of imaging parameters in an imaging condition of magnetic resonance imaging, and data indicative of a relationship of influence between the imaging parameters are stored. Based on a plurality of values corresponding to a plurality of imaging indices which relate to a plurality of imaging parameters and which influence magnetic resonance imaging, and based on the relationship of influence, a relationship diagram, which visually indicates the relationship between the plural values, is generated and displayed. Accordingly, at the time of editing the imaging condition, it is possible to intuitively present to the operator the influence which the edited imaging condition exerts on the imaging.

For example, in accordance with the moving operation of the point indicative of the value of the imaging index on the relationship diagram, or in accordance with the input of the value of the imaging parameter on the edit screen, the positions of the points indicative of the values of the imaging indices can be changed together with the relationship between the values of the imaging indices, and can be displayed on the relationship diagram, and the values of the imaging parameters on the edit screen can be changed and displayed. In addition, according to the MRI apparatus 1, the polygonal line, which corresponds to the base before the moving operation of the point indicative of the value of the imaging index, can be displayed before and after the edit of the imaging indices and imaging parameters. Moreover, according to the MRI apparatus 1, in accordance with the normalization instruction, the positions of the plural points indicative of the values of the imaging indices on the relationship diagram can be made uniform. Besides, according to the MRI apparatus 1, in accordance with the moving operation of the point indicative of the value of the imaging index on the relationship diagram, or the input of the value of the imaging parameter on the edit screen, the tentative image, which reflects the values of the imaging indices, can be generated and displayed together with the relationship diagram and the edit screen. For example, when any one of the imaging indices is an index relating to an artifact, and the value of this imaging index is a value indicative of the degree of the artifact, the influence, which the edit result on the other imaging indices exerts on the imaging index relating to the artifact, can be intuitively presented to the operator via the tentative image.

From the above, according to the MRI apparatus 1 relating to the present embodiment, in the edit of the imaging condition, the changes of the values of the plural imaging indices by the restrictions between the imaging parameters can be visually displayed on the relationship diagram, together with the relationship between the values of the imaging indices and the base, and the change of the relationship between the values of the imaging indices, relative to the base, can be intuitively presented to the operator. Thereby, it is possible to solve the problem that the influence on the imaging by the imaging condition after the edit of the imaging parameters is difficult to understand. For example, when imaging parameters, which are set for individual hospitals, are not proper imaging parameters depending on subjects, the imaging parameters need to be individually finely adjusted for each of the subjects. However, according to the present MRI apparatus 1, the influence on the imaging by the imaging condition can be intuitively presented to the operator, the re-setting of the imaging condition is made needless, and the edit efficiency of the imaging condition and the throughput of the MRI examination can be improved.

(Modification)

Compared to the second embodiment, the present modification is configured to restrict the movement of the point which co-varies in the radar chart. Accordingly, the input interface circuitry 29 inputs an instruction (restriction instruction) to restrict the influence between the imaging indices due to the movement of the point, with respect to at least one of the plural imaging indices displayed on the radar chart. The processing circuitry 33, which realizes the change function 337, changes the relationship of influence in accordance with the restriction instruction. The processing circuitry 33, which realizes the determination function 335, determines the values of the co-varying imaging indices, based on the changed relationship of influence and the change amount of the imaging parameter. The other configuration is the same as the configuration of the second embodiment.

The above-described restriction of movement is, for example, relaxation of movement and fixation. The relaxation of the influence between the imaging indices due to the movement of the point will be first described, and then the fixation of the point on the radar chart will be described.

(Relaxation of the Influence Between Imaging Indices)

The input interface circuitry 29 inputs, as a restriction instruction, a degree of priority which indicates the order of priority of relaxation of movement. As the degree of priority is lower, the influence between the imaging indices becomes smaller.

The processing circuitry 33, which realizes the determination function 335, determines the values of the imaging indices which co-vary with the movement, based on the change amounts of the imaging parameters due to the change of the value of the imaging index by the movement of the point, and the changed relationship of influence.

The display 31 displays, on the radar chart, a marker indicative of the degree of priority, at the point indicative of the value of the imaging index for which the degree of priority is designated.

The movement of the point relating to the imaging index, which is designated by the restriction instruction, becomes slow in the co-variance with the moving operation or the change operation. Specifically, the point relating to the imaging index, for which the restriction instruction is received, becomes less influenced by the moving operation or change operation, and thus becomes less easily movable on the radar chart. In addition, the moving operation for the imaging index, for which the restriction instruction is received, is suppressed, and the point relating to the imaging index, for which the restriction instruction is received, becomes difficult to move on the radar chart. The restriction instruction is useful when restrictions are to be imposed on the co-variance characteristic of the imaging index by the moving operation or change operation.

(Fixation of Imaging Index)

The input interface circuitry 29 inputs, as the restriction instruction, a fixing instruction to fix the point indicative of the value of the imaging index on the radar chart, with respect to at least one of the plural imaging indices displayed on the radar chart.

The processing circuitry 33, which realizes the determination function 335, determines the values of the co-varying imaging indices which are other than the imaging index designated by the fixing instruction, based on the change amounts of the imaging parameters due to the change of the value of the imaging index by the movement of the point, and the changed relationship of influence.

The display 31 displays, on the radar chart, a marker indicative of fixation, at the point indicative of the value of the imaging index designated by the fixing instruction.

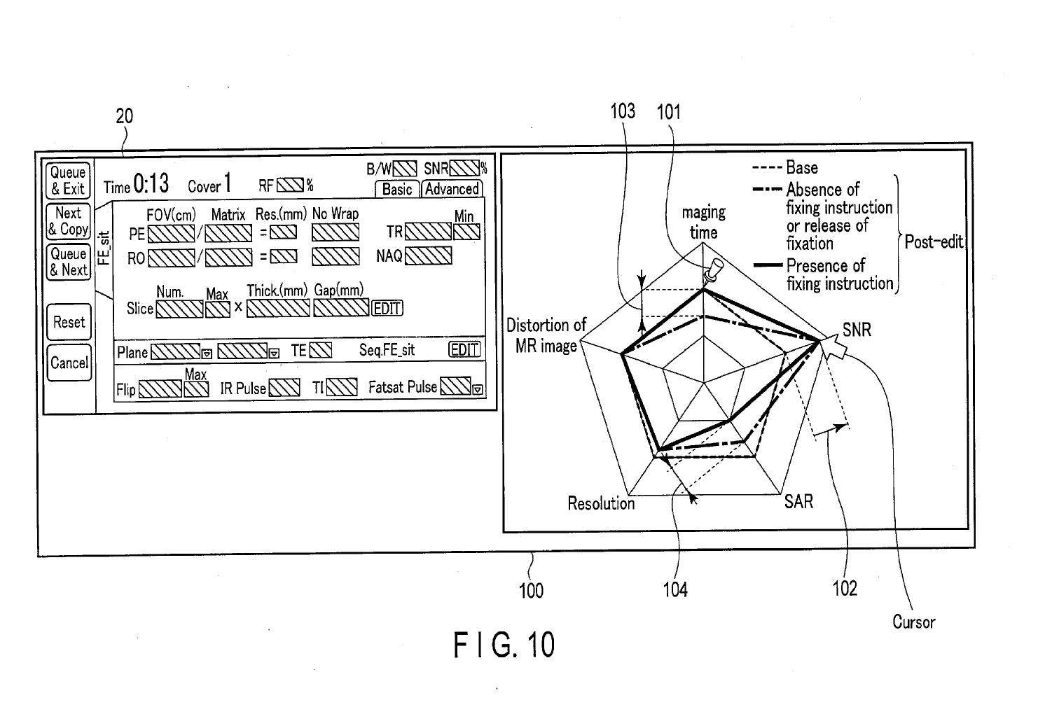

FIG. 10 is a view illustrating an example of a radar chart 100 in which the fixing instruction is input with respect to the imaging time. A marker 101 indicative of fixation is displayed as a pin-shaped image at the point indicative of the value of the imaging time in FIG. 10. As illustrated in FIG. 10, in a movement 102 of the point indicative of the value of the SNR, the point indicative of the value of the imaging time remains fixed. Reference numeral 103 in the radar chart 100 indicates a difference between the position of the point indicative of the value of the imaging time in the case of the absence of the fixing instruction, and the position of the point indicative of the value of the imaging time in the case of the presence of the fixing instruction, in the movement 102 of the point indicative of the value of the SNR. Reference numeral 104 in the radar chart 100 indicates a movement amount of the point indicative of the value of the SAR in the case in which the imaging time is fixed.

In the radar chart 100 in FIG. 10, when the point indicative of the value of the SNR is moved beyond a position indicative of a limit value of the imaging index (hereinafter referred to as “index limit position”), this point is moved back to the index limit position. Specifically, the movement of the point indicative of the value of the non-fixed imaging index is restricted by the index limit position. In the meantime, when the point indicative of the value of the SNR is moved beyond the index limit position, the marker 101 may be displayed in a different mode, for example, in a fallen pin shape, at a position apart from the position designated by the fixing instruction. At this time, the fixation of the point indicative of the value of the imaging time is released, and character information or the like notifying the release of fixation of the point indicative of the value of the imaging index may be displayed.

According to the above-described configuration, the following advantageous effects can be obtained.

According to the MRI apparatus 1 relating to the present modification, the restriction instruction to restrict the influence between the imaging parameters due to the movement of the point is input with respect to the imaging index displayed on the relationship diagram. The relationship of influence is changed in accordance with the restriction instruction, and the values of the imaging indices which co-vary with the movement, are determined based on the changed relationship of influence and the change amounts of the imaging parameters due to the change of the imaging index. Accordingly, in addition to the advantageous effects of the second embodiment, at the time of editing the imaging condition in the case in which the influence between the imaging indices is restricted, it is possible to intuitively present to the operator the influence which the edited imaging condition exerts on the imaging.

For example, in the relationship diagram, the movement of the point indicative of the value of the imaging index can be restricted. In addition, according to the present MRI apparatus 1, the marker can be displayed on the relationship diagram with respect to the imaging index for which the restriction of movement is designated. Thus, the operator can intuitively recognize, together with the relationship between the values of the imaging indices, the state of change of the points indicative of the values of the other imaging indices to which no priority is given.

In addition, according to the MRI apparatus 1, in the relationship diagram, the position of the point indicative of the value of the imaging index can be fixed such that this point does not move. Thereby, the operator can intuitively visually recognize, together with the relationship between the values of the imaging indices, the state of change of the other imaging parameters by the fixation of the imaging index. From these matters, according to the present MRI apparatus 1, a change of the imaging index, which the operator does not expect, can be prevented, re-setting of the imaging condition can be made needless, and the throughput of the edit of the imaging condition can be improved.

### Third Embodiment

Compared to the first embodiment and second embodiment, the present third embodiment is configured to move, on a relationship diagram, points indicative of values of a plurality of imaging indices which relate to a plurality of imaging parameters and which influence magnetic resonance imaging, and points indicative of values of a plurality of imaging parameters, and configured to display these points together with a relationship between the values of the imaging indices and imaging parameters. Accordingly, based on the plural values corresponding to the plural imaging indices which relate to the imaging parameters and which influence magnetic resonance imaging, and corresponding to the plural imaging parameters, and based on the relationship of influence, the processing circuitry 33, which realizes the relationship diagram generation function 333, generates a relationship diagram which visually indicates the relationship between the plural values. The display 31 displays the relationship diagram. The other configuration of the third embodiment is the same as the configurations of the first embodiment and second embodiment.

The input interface circuitry 29 inputs electric signals corresponding to a moving operation of a point indicative of the value of the imaging index on the relationship diagram, and a moving operation of a point indicative of the value of the imaging parameter on the relationship diagram.

The display 31 displays the relationship diagram which indicates the relationship between the plural values corresponding to the imaging indices which relate to the imaging parameters and which influence magnetic resonance imaging, and corresponding to the imaging parameters. In the meantime, the display 31 may display, together with the relationship diagram, a reference image or a tentative image, which reflects the values of the imaging indices and the values of the imaging parameters on the relationship diagram. Hereinafter, in order to make the description more concrete, it is assumed that a graph, which is used in the relationship diagram, is a radar chart. The number of axes of the radar chart corresponds to, for example, the number of imaging parameters and the number of imaging indices, which are selected by the operator. (Operation)

The operation according to the present embodiment is a combination of the operations according to the first and second embodiments. Specifically, the process procedure relating to the operation of this embodiment corresponds to a flowchart which is a combination of the flowcharts of FIG. 2, FIG. 3, FIG. 7 and FIG. 8. Thus, a description of the process procedure of the operation according to this embodiment is omitted.

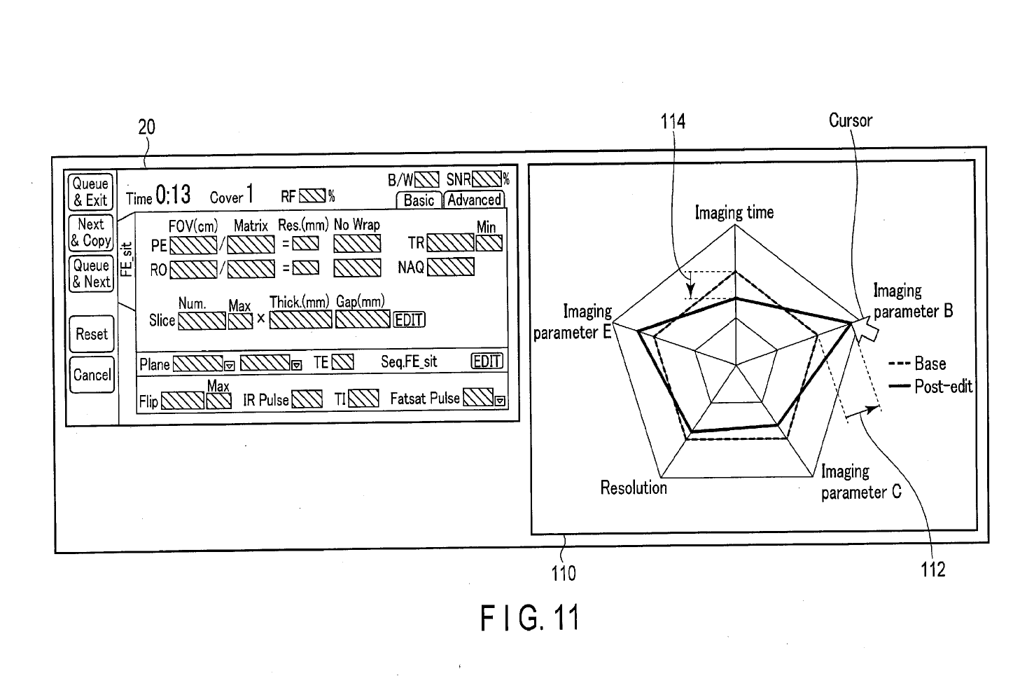

FIG. 11 is a view illustrating an edit screen 20 and a radar chart 110, which are displayed on the display 31. The imaging indices in the radar chart 110 are the resolution and imaging time, and the imaging parameters are imaging parameters B, C and E. The points indicative of the values before edit, which correspond to the resolution, imaging time, and imaging parameters B, C and E in the radar chart 110, are disposed equidistant from the center of the radar chart 110.

An arrow 112 shown in FIG. 11 indicates a change amount of the point indicative of the value of the imaging parameter B (a change amount of the imaging parameter B) on the radar chart 110. In the meantime, when at least two of the plural imaging indices and plural imaging parameters, which are displayed on the radar chart 110, are set as targets of the moving operation of points, the point indicative of the value of the set imaging index and the point indicative of the value of the set imaging parameter are moved together by the moving operation. Reference numeral 114 in FIG. 11 indicates a movement of the point indicative of the value of the imaging time, among the plural imaging indices which co-vary with the movement of the point indicative of the value of the imaging parameter B. As illustrated in FIG. 11, the points indicative of the values of the imaging parameter C, the resolution, and the imaging parameter E in the radar chart 110 are moved along the axes of the radar chart 110, in the co-variance with the movement of the point indicative of the value of the imaging parameter B. For example, a polygonal line that is a solid line in the radar chart 110 indicates the relationship between the values of the imaging indices and the values of the imaging parameters, which are changed in response to the movement of the point indicative of the value of the imaging parameter B. As illustrated in FIG. 11, the relationship between the values of the imaging indices and imaging parameters before and after the movement of the point, that is, the balance between the imaging indices and imaging parameters, is visualized by different display modes (e.g. pre-edit: dotted line, post-edit: solid line), and is displayed on the display 31.

According to the above-described configuration, the following advantageous effects can be obtained in addition to the advantageous effects described in the first embodiment and the second embodiment.

According to the MRI apparatus 1 relating to the present embodiment, a plurality of imaging parameters in an imaging condition of magnetic resonance imaging, and data indicative of a relationship of influence between the imaging parameters are stored. Based on the plural values corresponding to the plural imaging indices which relate to the imaging parameters and which influence magnetic resonance imaging, and corresponding to the plural imaging parameters, and based on the relationship of influence, the relationship diagram, which visually indicates the relationship between the plural values, is generated and displayed. Accordingly, at the time of editing the imaging condition, it is possible to intuitively present to the operator the influence which the edited imaging condition exerts on the imaging.

For example, in accordance with the moving operation of the points indicative of the plural values corresponding to the imaging indices and imaging parameters on the relationship diagram, the relationship between the plural values corresponding to the imaging indices and imaging parameters can be changed and displayed on the relationship diagram. Thereby, according to the MRI apparatus 1 relating to the present embodiment, in the edit of the imaging condition under the restrictions between the imaging parameters, the relationship between the plural values corresponding to the imaging indices and imaging parameters can be displayed on the relationship diagram, together with the base, and the change of the relationship can be intuitively presented to the operator.

(Modification)

Compared to the third embodiment, the present modification is configured to restrict the movement of the points indicative of the values of the imaging indices and imaging parameters which co-vary in the radar chart. Accordingly, the input interface circuitry 29 inputs an instruction (restriction instruction) to restrict the influence between the imaging parameters due to the movement of the points, with respect to the imaging indices and imaging parameters displayed on the radar chart. The processing circuitry 33, which realizes the change function 337, changes the relationship of influence in accordance with the restriction instruction. The processing circuitry 33, which realizes the determination function 335, determines the values of the co-varying imaging indices and imaging parameters, based on the changed relationship of influence and the change amount of the imaging parameter. The other configuration is the same as the configurations of the first to third embodiments. In addition, the contents of the configuration and operation in this modification correspond to the combination between the modification of the first embodiment and the modification of the second embodiment, so a description thereof is omitted.

According to the above-described magnetic resonance imaging apparatus 1, the plural imaging parameters in the imaging condition of magnetic resonance imaging, and data indicative of the relationship of influence between the imaging parameters are stored. Based on the plural values corresponding to at least either the plural imaging indices which relate to the imaging parameters and which influence magnetic resonance imaging, or the plural imaging parameters, and based on the relationship of influence, the relationship diagram, which visually indicates the relationship between the plural values, is generated and displayed. Thereby, at the time of editing the imaging condition, it is possible to intuitively present to the operator the influence which the edited imaging condition exerts on the imaging.

### FIRST APPLIED EXAMPLE

In the present applied example, on axes on a radar chart, clinically recommended ranges (hereinafter referred to as “adjustable ranges”) of values of imaging parameters are superimposed and displayed on the radar chart by a display mode such as half-tone dot meshing. In other words, ranges excluding the adjustable ranges in the radar chart correspond to clinically unrecommended ranges of values of imaging parameters. Thus, at the time of editing the imaging condition, the values of the imaging parameters are limited to the adjustable ranges. For example, in the radar chart 400 in FIG. 4, the adjustable range is displayed between the minimum value (the center of the radar chart), which the imaging parameter can take, and the maximum value (the outermost vertex of the radar chart) which the imaging parameter can take. The maximum value and minimum value of the imaging parameter in the radar chart correspond to, for example, limit values to hardware, such as hard limits of dB/dt. Incidentally, the hard limits may be associated with the maximum value and minimum value of the adjustable range.

In this applied example, in the change of the value of the imaging parameter according to the moving operation or change operation, the value of the imaging parameter after the change may be limited within the adjustable range. At this time, in the radar chart, the movement of the point indicative of the value of the imaging parameter is limited within the adjustable range. In the meantime, instead of displaying the adjustable range, the range of values, which the imaging parameter can take, may be limited within the adjustable range. Hereinafter, a process relating to this applied example will be described.

The processing circuitry 33, which realizes the determination function 335, sets the adjustable range for each of the imaging parameters, based on at least one of the pulse sequence in MR imaging (medical imaging), the purpose of imaging, the region of imaging, and subject information, and based on the relationship of influence between the imaging parameters. The subject information, for example, together with a patient ID, is transmitted to the MRI apparatus 1 from a radiology information system (RIS) or the like via a network (not shown). The subject information is, for example, the body height of the subject P, body weight, gender, body type, various information relating to implants, and various contraindication information relating to the subject P. The various information relating to implants is, for example, the position of an implant in the subject P, the kind of implant (pacemaker, artificial tooth, clip, stent, artificial valve, artificial inner ear, artificial joint, etc.), and the material of an implant (metal, silicone, etc.). In addition, the contraindication information is, for example, the length of gestation, presence/absence of a metallic piece in the body, presence/absence of a permanent tattoo, and presence/absence of various kinds of screws.

In the meantime, the adjustable range may be prestored in the storage 27 as a default, in accordance with the pulse sequence, purpose of imaging, region of imaging, and subject information. In addition, the adjustable range may be changed and input, as needed, by the operator's instruction via the input interface circuitry 29.

The processing circuitry 33, which realizes the change function 337, executes at least one of superimposing the adjustable range on the radar chart, and limiting the value of the imaging parameter within the adjustable range in the change of the value of the imaging parameter according to the moving operation or change operation. In the meantime, the processing circuitry 33 may change the adjustable range by using, for instance, the relationship of influence between the imaging parameters, in accordance with the change of the value of the imaging parameter according to the moving operation or change operation. The change of the adjustable range is, for example, the change of the width of the adjustable range on the axis of each imaging parameter in the radar chart. The change of the adjustable range will be described in an example which will be described later.

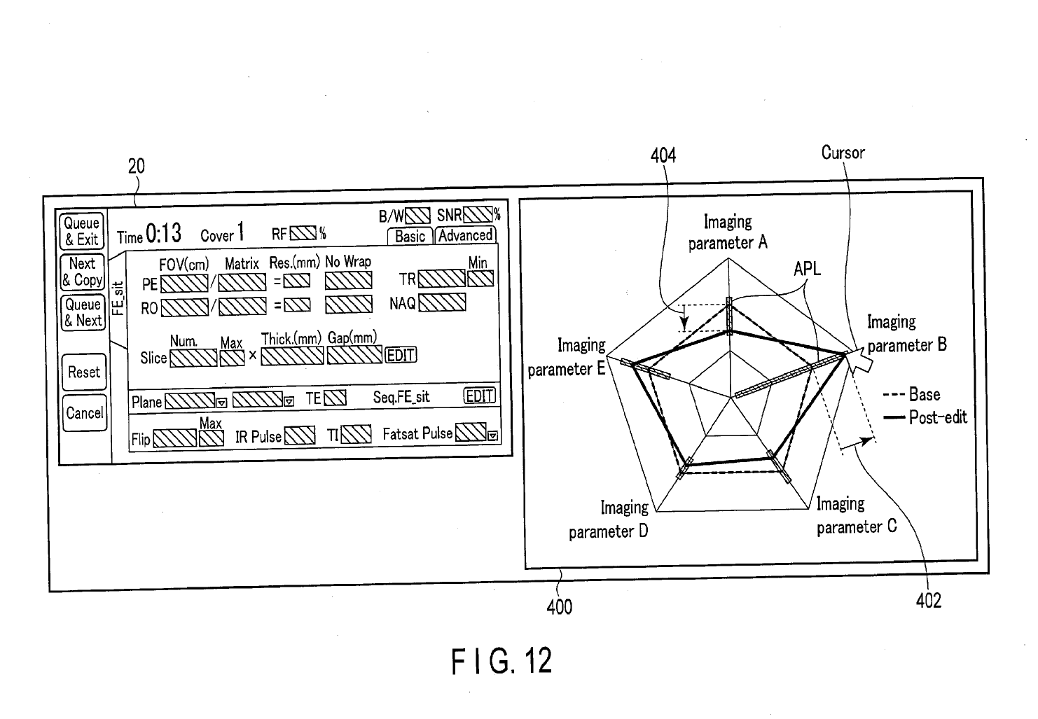

FIG. 12 is a view illustrating an example in which adjustable ranges are superimposed on a radar chart, in a case of using the radar chart as a relationship diagram. The radar chart in FIG. 12 corresponds to the radar chart 400 in FIG. 4. A plurality of half-tone dot meshings APL in FIG. 12 indicate adjustable ranges corresponding to the imaging parameters A, B, C, D and E.

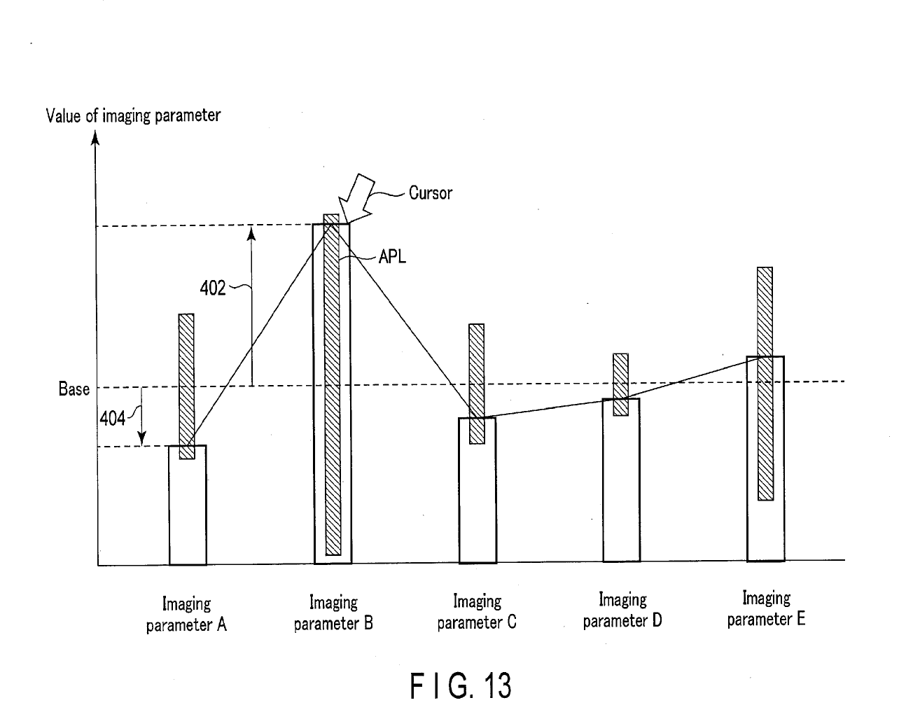

FIG. 13 is a view illustrating an example in which adjustable ranges are superimposed on a bar graph, in a case of using a bar graph as the relationship diagram. The bar graph in FIG. 13 corresponds to the radar chart 400 in FIG. 4. A sequential line, which connects vertices of a plurality of bars in the bar graph illustrated in FIG. 13, corresponds to the relationship between the values of five parameters after edit. A plurality of half-tone dot meshings APL in FIG. 13 indicate adjustable ranges corresponding to the imaging parameters A, B, C, D and E. An arrow 402 in FIG. 13 indicates a change amount of the imaging parameter B in the bar graph. An arrow 404 in FIG. 13 indicates a change in height of the bar indicative of the value of the imaging parameter A, among the plural imaging parameters which co-vary with the change of the height of the bar indicative of the value of the imaging parameter B. As illustrated in FIG. 13, the heights of the bars indicative of the values of the imaging parameters A, C, D and E are changed in the co-variance with the change of the height of the bar indicative of the value of the imaging parameter B. A sequential line that is a solid line in the bar graph of FIG. 13 indicates the relationship between the values of the imaging parameters, which are changed in response to the change of the height of the bar indicative of the value of the imaging parameter B. As illustrated in FIG. 13, the relationship between the values of the imaging indices before and after the change of the height of the bar, that is, the balance between the plural imaging parameters, is visualized by different display modes (e.g. pre-edit: dotted line, post-edit: solid line), and is displayed on the display 31.

In the above description of the present applied example, the imaging parameters are used. However, this applied example may be implemented for the imaging indices, or may be implemented for the combination of the imaging parameters and imaging indices. Since the adjustable ranges relating to the imaging indices can be understood by reading the term “imaging parameters” in the above description as “imaging indices”, a description of such adjustable ranges is omitted. Hereinafter, a description will be given of an example of the case in which the imaging parameters and imaging indices are mixedly present.

In the relationship diagram, when the SAR is displayed as the imaging index and the dB/dt and TR are displayed as the imaging parameters, if the point indicative of the value of the SAR is lowered (decrease of SAR), the point indicative of the dB/dt is unchanged, and the point indicative of the TR is moved to a position indicative of a large value within the adjustable range.

As regards the change of the adjustable ranges, a description will be given of, by way of example, the case in which the image quality is displayed as the imaging index and the acceleration factor is displayed as the imaging parameter on the radar chart as illustrated in FIG. 11. At this time, it is assumed that the default adjustable range relating to the acceleration factor is from 2 to 8. In addition, in this radar chart, when the point indicative of the value of the image quality is moved toward a higher image quality by the moving operation, the adjustable range relating to the acceleration factor is changed from 2 to 4. Specifically, the adjustable range is changed in consideration of the property of the imaging parameter in relation to the imaging index, based on the relationship of influence between the plural imaging parameters and the conversion table.

According to the above-described configuration, the following advantageous effects can be obtained in addition to the advantageous effects described in the other embodiments.

According to the MRI apparatus 1 relating to the present applied example, it is possible to execute at least one of superimposing on the relationship diagram the adjustable ranges indicative of the clinically recommended ranges with respect to the values of the imaging parameters, and limiting the values of the imaging parameters within the adjustable ranges in the change of the value of the imaging parameter according to the moving operation or change operation. In addition, according to the MRI apparatus 1 relating to this applied example, it is possible to execute at least one of superimposing on the relationship diagram the adjustable ranges indicative of the clinically recommended ranges with respect to the values of the imaging indices, and limiting the values of the imaging indices within the adjustable ranges in the change of the value of the imaging index according to the moving operation or change operation. Thereby, according to the MRI apparatus 1 relating to this applied example, since the imaging condition can edited within the clinically recommended adjustable ranges, the edit efficiency of the imaging condition and the throughput of the MRI examination can be improved.

### SECOND APPLIED EXAMPLE

For example, in the present applied example, a plurality of relationship diagrams corresponding to stages such as a subject information, a pulse sequence in MR imaging, a region of imaging, an imaging protocol, and a preset are stored. Based on the preset or imaging protocol selected by an operator's instruction, the relationship diagram of a display target is specified from the plural relationship diagrams, and the specified relationship diagram is displayed on the display 31 at the time of editing imaging conditions. Plural imaging parameters, which are displayed on the stored relationship diagrams, are a part of many imaging parameters relating to subject information, a pulse sequence in MR imaging, a region of imaging, an imaging protocol, a preset, etc. and are set as defaults in advance. The imaging parameters for each stage in relationship diagrams are pre-settable by operators, etc. Also, the imaging parameters are settable for any stage in relationship diagrams as desired by operators, etc.

The storage 27 stores the plural relationship diagrams which are generated in advance by the relationship diagram generation function 333 in accordance with the subject information, the pulse sequences in MR imaging, the regions of imaging, imaging protocols, and presets. Specifically, each of the plural relationship diagrams is a typical relationship diagram which is generated prior to the edit of the imaging condition, with respect to each of subjects, each of imaging protocols, each of regions of imaging, each of the pulse sequences, and each of presets.

The processing circuitry 33 specifies, by the determination function 335, the relationship diagram of the display target from among the plural relationship diagrams by a statistical process such as a maximum likelihood method, based on the preset or imaging protocol selected by the operator's instruction. In the meantime, the processing circuitry 33 may specify the relationship diagram by using examination information or subject information, which is transmitted from the RIS or the like to the MRI apparatus 1. For example, the processing circuitry 33 specifies a relationship diagram, which most agrees with the selected preset or imaging protocol, and with the subject information, as the relationship diagram which is displayed at the time of editing the imaging condition. The processing circuitry 33 displays the specified relationship diagram on the display 31.

As an example of the specifying of the relationship diagram, a description will be given of a case in which an implant is applied to the subject P of the imaging target. Based on the information relating to the implant in the subject information, the processing circuitry 33 specifies, among the plural relationship diagrams stored in the storage 27, the relationship diagram which corresponds to the selected preset or imaging protocol and relates to the implant. The specified relationship diagram is, for example, a radar chart in which the values of imaging parameters are restricted to those for the implant. In the above description of the present applied example, the imaging parameters are used. However, this applied example may be implemented for the imaging indices, or may be implemented for the combination of the imaging parameters and imaging indices.

According to the above-described configuration, the following advantageous effects can be obtained in addition to the advantageous effects described in the other embodiments.

According to the MRI apparatus 1 relating to the present applied example, the plural relationship diagrams which are set in accordance with the regions of imaging in the medical imaging, the pulse sequences, the subject information, imaging protocols and presets, are stored. Based on the preset or imaging protocol selected by the operator's instruction, a relationship diagram of a display target is specified from the plural relationship diagrams, and the specified relationship diagram can be displayed at the time of editing the imaging condition. Thereby, according to the MRI apparatus 1 relating to this applied example, it is possible to prepare the plural relationship diagrams which are generated in advance in accordance with the regions of imaging in the medical imaging, sequences, subject information, imaging protocols and presets, and to specify and display, from among the prepared relationship diagrams, the relationship diagram which corresponds to the preset or imaging protocol selected by the operator's instruction, and to the subject information. According to this applied example, based on the imaging protocol or the preset selected by the operator, the optimal relationship diagram for the edit of the imaging condition can be specified and displayed, from among the typical relationship diagrams which are generated in advance. Therefore, the edit efficiency of the imaging condition and the throughput of the MRI examination can be improved.

### THIRD APPLIED EXAMPLE

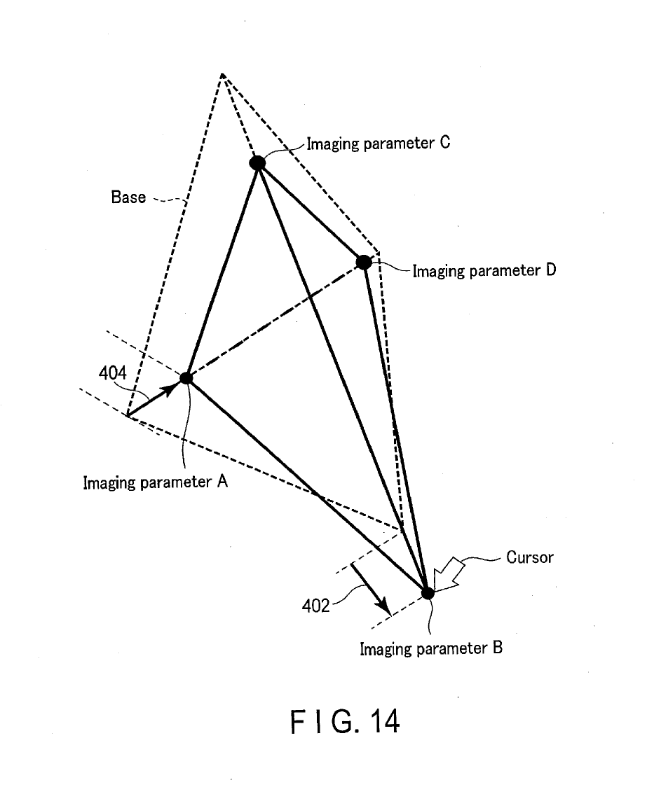

In the present applied example, a three-dimensional polygon, such as a polyhedron, is used as the relationship diagram. In this case, the imaging parameters and imaging indices are disposed at vertices of the three-dimensional polyhedron. The polyhedron, which is displayed on the display 31, is rotated as needed by the operator's instruction through the input interface circuitry 29. Hereinafter, the present applied example will be described with reference to FIG. 14.

FIG. 14 is a view illustrating an example of a case of using a tetrahedron as the relationship diagram. The tetrahedron in FIG. 14 corresponds to the radar chart 400 in FIG. 4, from which the imaging parameter E is excluded. A plurality of sides of the tetrahedron illustrated in FIG. 14 correspond to the relationship between the values of the four imaging parameters after edit. An arrow 402 shown in FIG. 14 indicates a change amount of the imaging parameter B in the tetrahedron. An arrow 404 shown in FIG. 14 indicates a movement of the point indicative of the value of the imaging parameter A, among the plural imaging parameters which co-vary with the change of the position of the point indicative of the value of the imaging parameter B. As illustrated in FIG. 14, the points indicative of the values of the imaging parameters A, C and D are changed in the co-variance with the movement of the point indicative of the value of the imaging parameter B. A solid line in the tetrahedron in FIG. 14 indicates the relationship between the values of the imaging parameters, which are changed in response to the movement of the point indicative of the value of the imaging parameter B. As illustrated in FIG. 14, the relationship between the values of the imaging parameters before and after the movement of the point, that is, the balance between the plural imaging parameters, is visualized by different display modes (e.g. pre-edit: dotted line, post-edit: solid line), and is displayed on the display 31.

According to the present applied example, when it is difficult for the operator to understand the change of the relationship between the plural values of the imaging parameters and the change of the relationship between the plural values of the imaging indices due to the increase of the imaging parameter and the increase of the imaging index displayed on the relationship diagram, the influence, which the edited imaging condition exerts on the imaging, can be intuitively presented to the operator.

### FOURTH APPLIED EXAMPLE

The technical concept of the magnetic resonance imaging apparatus 1 can be realized by various modalities (medical image diagnostic apparatuses) such as an X-ray computed tomography (CT) apparatus, an X-ray diagnostic apparatus, an ultrasonic diagnostic apparatus and a nuclear medical diagnostic apparatus. The medical image diagnostic apparatus includes the respective structural components in a dot-and-dash box 50 in FIG. 1.

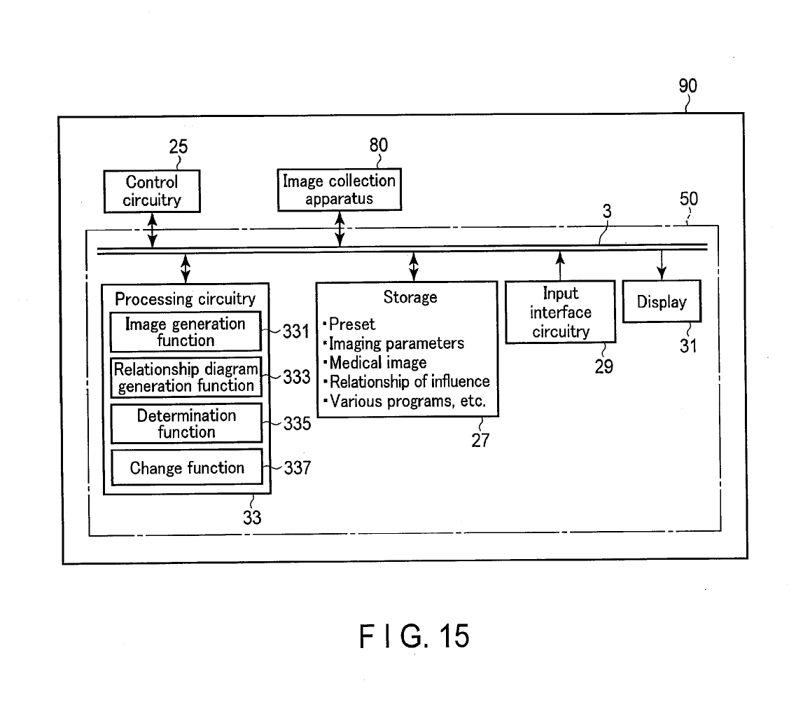

FIG. 15 is a block diagram illustrating an example of the configuration of the medical image diagnostic apparatus. As shown in FIG. 15, a medical image diagnostic apparatus 90 includes control circuitry 25 and an image collection apparatus 80, in addition to the respective structural components in the dot-and-dash box 50 in FIG. 1. For example, if this medical image diagnostic apparatus is an X-ray CT apparatus or a nuclear medical diagnostic apparatus, the image collection apparatus 80 includes various structural components mounted on a gantry. If the medical image diagnostic apparatus is an X-ray diagnostic apparatus, the image collection apparatus 80 corresponds to an X-ray imaging system including an X-ray tube, X-ray detector, etc. If the medical image diagnostic apparatus is an ultrasonic diagnostic apparatus, the image collection apparatus 80 corresponds to an ultrasonic imaging system including an ultrasonic probe, etc.

The medical image diagnostic apparatus executes the various processes described in connection with the above-described image generation function 331, relationship diagram generation function 333, determination function 335 and change function 337, in accordance with the imaging condition, imaging parameters, imaging indices, etc., which are unique to the various modalities. Hereinafter, the X-ray CT apparatus will be described as an example of the medical image diagnostic apparatus.

The X-ray CT apparatus executes, in some cases, photography of the heart of the subject P by helical scan, with the presupposition of segment reconstruction. In the photography of the heart by the X-ray CT apparatus, there is known a technique in which a proper scan speed and helical pitch are calculated in accordance with the heart beat information of the subject P, and thereby an optimal temporal resolution is obtained. The scan speed corresponds to, for example, a rotational speed of a rotary frame on which an X-ray tube and an X-ray detector are mounted. In this technique, the imaging parameters relating to the imaging condition, which is associated with the photography of the heart, are the scan speed and helical pitch. In addition, the temporal resolution as the imaging index relating to the photography of the heart is associated with the scan speed and helical pitch. When the imaging condition is edited, the scan speed, helical pitch and temporal resolution, which are mutually associated, are displayed on the display 31 such that the scan speed, helical pitch and temporal resolution can be changed on the radar chart in the co-varying manner.

The advantageous effects of the present applied example are the same as those in the first to third embodiments, so a description thereof is omitted.

According to the medical image diagnostic apparatuses of the above-described embodiments, modifications and applied examples, when the imaging condition is edited, the influence, which the edited imaging condition exerts on imaging, can be intuitively presented to the operator.

While certain embodiments have been described, these embodiments have been presented by way of example only, and are not intended to limit the scope of the inventions. Indeed, the novel embodiments described herein may be embodied in a variety of other faints; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image diagnostic apparatus comprising:
a display configured to display a relationship diagram indicating a relationship between a plurality of values which are respectively set for a plurality of imaging parameters relating to an imaging condition of medical imaging; and
processing circuitry configured to
update the relationship diagram in accordance with a moving operation of a point corresponding to each of the values on the relationship diagram, or a change operation of the imaging condition, and
change the imaging condition before the moving operation or before the change operation to an imaging condition which reflects the moving operation or the change operation.

2. The medical image diagnostic apparatus according to claim 1, further comprising a storage configured to store the imaging parameters, and data indicative of a relationship of influence between the imaging parameters,
wherein the processing circuitry is configured to generate the relationship diagram, based on the values and the relationship of influence.

3. The medical image diagnostic apparatus according to claim 1, wherein the display is configured to display the relationship diagram together with an edit screen of the imaging parameters.

4. The medical image diagnostic apparatus according to claim 2, wherein
the processing circuitry is configured to
determine, in the relationship diagram, the values of the imaging parameters which co-vary with the moving operation or the change operation, based on a change amount of the imaging parameter by the moving operation or the change operation, and based on the relationship of influence, and
update the relationship diagram by changing the relationship with use of the determined values, and
the display is configured to display the updated relationship diagram.

5. The medical image diagnostic apparatus according to claim 4, wherein the processing circuitry is configured to
change the relationship of influence with respect to the imaging parameters displayed on the relationship diagram, in accordance with a restriction instruction to restrict an influence between the imaging parameters by the moving operation, and
determine the values of the co-varying imaging parameters, based on the changed relationship of influence and the change amount.

6.. The medical image diagnostic apparatus according to claim 4, wherein the processing circuitry is configured to change an imaging condition which is commonly included in a plurality of imaging protocols that are associated by using a pulse sequence, a purpose of imaging and a region of imaging, to an imaging condition which reflects the change amount and the relationship of influence.

7. The medical image diagnostic apparatus according to claim 1, wherein the processing circuitry is configured to generate the relationship diagram in accordance with at least one of selection of the imaging parameters which are displayed on the relationship diagram, and selection of a graph which is used as the relationship diagram.

8. The medical image diagnostic apparatus according to claim 1, further comprising input interface circuitry configured to set, as targets of the moving operation, at least two of the imaging parameters in the relationship diagram.

9. The medical image diagnostic apparatus according to claim 1, further comprising input interface circuitry configured to input a normalization instruction to make uniform the points indicative of the values of the imaging parameters in the relationship diagram,
wherein the processing circuitry is configured to uniformize positions of the points indicative of the values of the imaging parameters by changing scale of the imaging parameters in the relationship diagram in response to the normalization instruction.

10. The medical image diagnostic apparatus according to claim 1, wherein the processing circuitry is configured to generate a tentative image corresponding to the imaging condition, in accordance with the moving operation, and
the display is configured to display the tentative image together with the relationship diagram.

11. The medical image diagnostic apparatus according to claim 1, wherein the processing circuitry is configured to execute at least one of superimposing, on the relationship diagram, adjustable ranges indicative of clinically recommended ranges with respect to the values of the imaging parameters, and limiting the values within the adjustable ranges in changing the values of the imaging parameters according to the moving operation or the change operation.

12. The medical image diagnostic apparatus according to claim 1, further comprising a storage configured to store a plurality of the relationship diagrams which are generated in accordance with a region of imaging in the medical imaging, a sequence, subject information, an imaging protocol, and a preset in which a plurality of the imaging protocols each including the imaging condition corresponding to the region of imaging and a purpose of imaging are set in a desired order,
wherein the processing circuitry is configured to specify a relationship diagram of a display target from among the plurality of relationship diagrams, based on the preset or the imaging protocol, which is selected by an instruction of an operator, and
the display is configured to display the specified relationship diagram.

13. A medical image diagnostic apparatus comprising:
a display configured to display a relationship diagram indicating a relationship between a plurality of values that are respectively set for a plurality of imaging indices which relate to a plurality of imaging parameters relating to an imaging condition of medical imaging and which indicate influence exerted on the medical imaging; and
processing circuitry configured to
update the relationship diagram in accordance with a moving operation of a point corresponding to each of the values on the relationship diagram, or a change operation of the imaging condition, and
change the imaging condition before the moving operation or before the change operation to an imaging condition which reflects the moving operation or the change operation.

14. The medical image diagnostic apparatus according to claim 13, further comprising a storage configured to store the imaging parameters, and data indicative of a relationship of influence between the imaging parameters,
wherein the processing circuitry is configured to generate the relationship diagram, based on the values and the relationship of influence.

15. The medical image diagnostic apparatus according to claim 13, wherein the display is configured to display the relationship diagram together with an edit screen of the imaging parameters.

16. The medical image diagnostic apparatus according to claim 14, wherein
the processing circuitry is configured to
determine, in the relationship diagram, the values of the imaging indices which co-vary with the moving operation or the change operation, based on a change amount of the imaging index by the moving operation or a change amount of the imaging parameter by the change operation, and based on the relationship of influence, and
update the relationship diagram by changing the relationship with use of the determined values, and
the display is configured to display the updated relationship diagram.

17. The medical image diagnostic apparatus according to claim 16, wherein the processing circuitry is configured to
change the relationship of influence with respect to the imaging indices displayed on the relationship diagram, in accordance with a restriction instruction to restrict an influence between the imaging indices by the moving operation, and
determine the values of the co-varying imaging indices, based on the changed relationship of influence and the change amount.

18. The medical image diagnostic apparatus according to claim 14, wherein the processing circuitry is configured to determine a value of a new imaging index by using the values of the plurality of imaging parameters designated by an operator, and the relationship of influence.

19. The medical image diagnostic apparatus according to claim 16, wherein the processing circuitry is configured to change an imaging condition which is commonly included in a plurality of imaging protocols that are associated by using a pulse sequence, a purpose of imaging, a region of imaging and subject information, to an imaging condition which reflects the change amount and the relationship of influence.

20. The medical image diagnostic apparatus according to claim 13, wherein the processing circuitry is configured to generate the relationship diagram in accordance with at least one of selection of the imaging indices which are displayed on the relationship diagram, and selection of a graph which is used as the relationship diagram.

21. The medical image diagnostic apparatus according to claim 13, further comprising input interface circuitry configured to set, as targets of the moving operation, at least two of the imaging indices in the relationship diagram.

22. The medical image diagnostic apparatus according to claim 13, further comprising input interface circuitry configured to input a normalization instruction to make uniform the points indicative of the values of the imaging indices in the relationship diagram,
wherein the processing circuitry is configured to uniformize positions of the points indicative of the values of the imaging indices by changing scale of the imaging indices in the relationship diagram in response to the normalization instruction.

23. The medical image diagnostic apparatus according to claim 13, wherein the processing circuitry is configured to generate a tentative image corresponding to the imaging condition, in accordance with the moving operation, and
the display is configured to display the tentative image together with the relationship diagram.

24. The medical image diagnostic apparatus according to claim 13, wherein the imaging indices include an index relating to an artifact, and
the value of the imaging index is a value indicative of a degree of the artifact.

25. The medical image diagnostic apparatus according to claim 13, wherein the processing circuitry is configured to execute at least one of superimposing, on the relationship diagram, adjustable ranges indicative of clinically recommended ranges with respect to the values of the imaging indices, and limiting the values within the adjustable ranges in changing the values of the imaging indices according to the moving operation or the change operation.

26. The medical image diagnostic apparatus according to claim 13, further comprising a storage configured to store a plurality of the relationship diagrams which are generated in accordance with a region of imaging in the medical imaging, a sequence, subject information, an imaging protocol, and a preset in which a plurality of the imaging protocols each including the imaging condition corresponding to the region of imaging and a purpose of imaging are set in a desired order,
wherein the processing circuitry is configured to specify a relationship diagram of a display target from among the plurality of relationship diagrams, based on the preset or the imaging protocol, which is selected by an instruction of an operator, and
the display is configured to display the specified relationship diagram.
